(19) United States
(12) Patent Application Publication
(10) Pub. No.: US 2010/0146662 A1
(43) Pub. Date: Jun. 10, 2010

(54) USE OF R-GENES AS A SELECTION MARKER IN PLANT TRANSFORMATION AND USE OF CISGENES IN PLANT TRANSFORMATION

(76) Inventors: Nicolaas Clemens Maria Henricus de Vetten, Groningen (NL); Richard Gerardus Franciscus Visser, Bennekom (NL); Evert Jacobsen, Wageningen (NL); Edwin Andries Gerard van der Vossen, Utrecht (NL); Anna Maria Agnes Wolters, Wageningen (NL)

Correspondence Address: HOFFMANN & BARON, LLP, 6900 JERICHO TURNPIKE, SYOSSET, NY, 11791, US

(21) Appl. No.: 12/523,795

(22) PCT Filed: Jan. 28, 2008
(86) PCT No.: PCT/NL08/50051

§ 371 Date: Nov. 16, 2009

(30) Foreign Application Priority Data
Jan. 26, 2007 (EP) 07101275.1

(51) Int. Cl. A01H 1/00; A61K 49/00; A01H 5/00; A61P 43/00

(52) U.S. Cl. 800/278; 424/9.1; 800/295

(57) ABSTRACT

The invention relates to the field of plant transformation, in particular plant transformation of a Solanaceae, preferably of potato. The present invention provides an alternative selection method in plant transformation processes. The invention further provides a plant that has been provided with additional nucleic acid sequences but which genetically modified plant essentially consists of cis plant sequences, for example a genetically modified potato plant that has been provided with additional (essentially) potato plant sequences. Such a transgenic plant is free from non-Solanum T-DNA border sequences. A preferred embodiment is a potato plant that carries a functional R-gene, providing resistance against an oomycete pathogen, preferably Phytophthora infestans, wherein said R-gene can be used as selectable marker.

The invention relates to the field of plant transformation, in particular plant transformation of a Solanaceae, preferably of potato.

Transformation of plants using transforming bacteria such as Agrobacterium spp. to obtain transgenic plants expressing a heterologous gene or a gene fragment of interest has been known since the late seventies and early eighties of the last century when it was found that the Ti (tumor inducing) plasmid of said bacterium could be used as a vector in genetic engineering of plants. The wild-type plasmid induces plant cells to produce tumor cells, but it can be modified so that it can carry foreign gene constructs into cells without necessarily making the recipient cells tumorous. During tumor induction, a specific segment of the Ti plasmid, called the T-DNA (transferred DNA), integrates into the host plant nuclear DNA. In genetic engineering of plants, said T-DNA is modified and now carries the foreign gene construct to be integrated in the plant's DNA so that a transgenic plant can be obtained.

A transformation procedure for obtaining transgenic plants generally consists of infection of a plant cell with a transforming bacterium, which generally comprises an essentially non-tumorigenic Agrobacterium strain, which bacterium is provided with a recombinant nucleic acid comprising a T-DNA vector construct allowing for transfer of said construct into the genome of a plant cell, said construct essentially comprising the desired nucleic acid, gene or gene fragment that one wishes to see expressed in a finally transformed plant and a selective marker nucleic acid or selection gene. This desired heterologous gene and the marker are in general located on a plasmid or vector in a piece of the T-DNA, which is the DNA flanked by at least one, or located between two imperfect direct repeats of most often 24 basepairs length, called the T-DNA borders. Transfer of the heterologous gene/selection gene construct into the plant cell takes place in a process whereby bacterial vir genes (located on the same or different plasmid) are involved to accommodate transfer and integration of the T-DNA/gene construct. Vir-proteins (D1 and D2) cause nicking of the border repeats at a precise site whereby the T-DNA construct is cut at the T-DNA borders from the plasmid and inserted into the plant genome.

By growing the thus treated plant cells in an appropriate environment, whole plants can be regenerated, some of which carry the desired gene. To select for the desired transformed cells in the background of untransformed cells DNA encoding said selection gene and means for its expression is generally physically linked to the gene of interest on the T-DNA to permit the recovery of transformants. The presence of such a selection or marker sequence in the T-DNA is generally seen as an absolute requirement for efficient recovery; otherwise tenths- to hundreds-of-thousands putative transformants would need to be screened for the presence and functional insertion of the desired heterologous gene. Instead, putative transformants are almost always grown in a selective medium or under selective pressure appropriate for the selective marker chosen to give the transformed cells a selective advantage over the, initially over-abundantly present, non-transformed cells.

Historically, a plant selection marker is a dominant gene that, after expression, confers resistance to a selective agent that is added to the regeneration medium, but which itself is not essential for cell growth. Such a selective agent is for example an antibiotic, herbicide, amino acid or amino acid analog added to a plant or plant culturing medium in a toxic concentration. Among the selective markers or selection genes that are most widely used in plant transformation are the bacterial neomycin phosphotransferase genes (nptI, nptII and nptIII genes) conferring resistance to the selective agent kanamycin, suggested in EP131623 and the bacterial aphIV gene suggested in EP186425 conferring resistance to hygromycin. EP 275957 discloses the use of an acetyl transferase gene from Streptomyces viridochromogenes that confers resistance to the herbicide phosphinotricin. Plant genes conferring relative resistance to the herbicide glyphosate are suggested in EP218571. The resistance is based on the expression of a gene encoding 5-enolshikimate-3-phosphate synthase (EPSPS) that is relatively tolerant to N-phosphomethylglycine. Certain amino acids such as lysine, threonine, or the lysine derivative amino ethyl cysteine (AEC) and tryptophan analogs like 5-methyl tryptophan can also be used as selective agents due to their ability to inhibit cell growth when applied at high concentration. In this selection system expression of the selectable marker gene results in overproduction of amino acids by transgenic cells which permits the transgenic to grow under selection.

Another class of selectable markers are those that support growth and proliferation of the transformed plant cells under conditions that are insufficient for the growth of non-transformed cells, e.g. conditions lacking a plant growth hormone. A selection marker gene can encode an enzyme that after expression converts an encryptic carbon source into a carbon source that supports growth and proliferation of the transformed plant cells under conditions that contain minimal nutrients and in which the carbon source is replaced by an encryptic or latent carbon source that cannot be utilized by non-transformed cells. An example of such a positive selection marker is the phosphomannose isomerase that converts non-utilizable mannose-6-phosphate into fructose-6-phosphate that can be used by plant cells as a carbon source (suggested in U.S. Pat. No. 6,143,562) or xylose isomerase from Streptomyces rubiginosus (Haldrup A., et al. 1998, Plant Cell Report 18:76-81).

Yet another class of selection marker genes allows the screening of presumably transformed plant cells rather than direct genetic selection of transformed cells for resistance to a toxic substance such as an antibiotic. For this reason they are often also called screenable markers. The genes are referred to as reporter genes and include for instance beta-glucuronidase (GUS), beta-galactosidase, luciferase, and green fluorescent protein (GFP).

Alternatively, a screenable marker may provide some other visible reactive response. For instance, it may cause a distinctive appearance or growth pattern relative to plants or plant cells not expressing a screenable marker gene in the presence of a substance, which can either be applied to the plant or plant cells directly or a substance which is present in the plant or in the plant cell growth media.

Generally, the plants or plant cells containing such screenable marker genes have a distinctive phenotype for purpose of identification, i.e., they can be distinguished from non-transformed cells. The characteristic phenotype allows the identification of cells, cell groups, tissues, organs, plant parts or whole plants containing the construct. An example of a morphological abnormality induction (MAI) marker gene is the isopentenyl transferase gene from Agrobacterium (Keller et al. WO 00/37060). Isopentenyl transferase is a rate-limiting enzyme in the biosynthesis of cytokinin, which is a plant growth hormone. A plant cell into which the ipt gene is introduced produces cytokinin, with the result that the proliferation and differentiation of the cell containing the ipt gene are confused to induce various morphological abnormalities.

The presence of non-endogenous sequences such as the Agrobacterium T-borders and antibiotic resistance genes and other selective markers sequences in the final transgenic plant obtained is in most cases considered undesirable. These sequences, albeit thought to be necessary for the transformation processes, do in general not positively contribute to the final transformed plant and in fact lessen its desirability to the consumer for a number of reasons. Consumer groups express concern about the widespread distribution of resistance markers in food products referring to a theoretical risk of a horizontal transfer of transgene selection genes into gut bacteria.

Environmental groups are concerned about the risk of cross-pollination between the transgenic plant and related species which can lead to a transfer of resistance traits into weeds, jeopardizing the long-term use of transgenic crops and causing potential ecological problems.

A number of systems have so far been developed to facilitate the removal of selective marker genes. Co-transformation of two different constructs can result in transgenic lines that have integrated both transgenes and after a genetic cross the selective marker can be segregated away from the gene of interest. Removal of selective marker genes by co-transformation is suggested in e.g. WO95/16031, U.S. Pat. No. 6,265,638 and WO00/18939. A co-transformation system however requires that the marker gene is located at an unlinked location meaning screening of many more independent transformation events. Moreover, many cross pollinating and in particular vegetatively propagated crops (like apple or strawberry), and especially tuberous crops like potato and cassava, are highly heterozygous and removal of the marker sequence by genetic segregation would require many years to find a clone with suitable field performance. Several transposable element systems and site-specific recombination systems have been employed for marker removal (Sugita K, et al. 2000 Plant J 22: 461-469; Zuo J, et al. 2001 Nature Biotech 19: 157-161). EP 716147 describes a vector for introducing a desired gene into plants, comprising the gene of interest and at least a morphological abnormality induction (MAI) marker gene placed on a removable DNA element i.e. a transposable element or site-specific recombination system. Plants transformed with the MAI containing gene construct can be easily detected by eye by their abnormal morphology of the shoots. Likewise, the loss of the MAI gene's function after transposition of the transposable element or after site-specific removal of the recombination system can be easily detected as this results in normal looking shoots. Such transgenic plants can be produced free of marker genes without having to undergo the crossing step. These systems require the expression of a transposase or recombinase that mediates the deletion of regions bracketed between recombination or transposase target sequences, and the subsequent removal of the marker gene by genetic segregation. Also these systems are time consuming as well as impractical for species that are mainly propagated vegetatively. Moreover, deleted fragments can reinsert into other genomic positions. Another approach to induce DNA deletions is based on intrachromosomal homologous recombination between two homologous sequences. Intrachromosomal homologous recombination frequencies are often too low for an efficient application of this system to produce deletions of transgene regions. Using the attP region of bacteriophage λ this recombination frequency could be increased (Zubko E, et al. 2000 Nature Biotech 18: 442-445). However, the process is still unpredictable and too inefficient to generate hundreds to thousands of independent transformation events to find the clone with suitable agronomical performance. A system to recover transgenic cells without the use of selective markers has been described. In WO98/51806 a method is disclosed for the recovery of transformed cells without the use of selectable markers by enrichment of transgenic sectors using nodal culture and non-selective screening assays. This method involves the culturing of the transformed plant cells or tissue comprising a non-selectively assayable transgene until nodes comprising meristematic tissue have developed. Subsequently, the plant tissue is assayed using a non-selective assay, such as enzyme assays or ELISA's. Assay-positive plants that are recovered with this method are chimeric and have transformed sectors. To enrich these transformed sectors from the assay-positive tissue nodal explants are prepared and cultured such that shoots are formed. Shoots and leaves are to be assayed again using a non-selective assay in the hope that plants are recovered with enriched transformed sectors so that eventually, after several rounds of assaying, near-uniform transgenic plants can be obtained.

WO 03/010319 describes a method for obtaining marker-free plants comprising a recombinant nucleic acid comprising a T-DNA construct allowing for transfer of said construct into the genome of a plant cell, said construct provided with a foreign nucleic acid that is free of nucleic acid encoding a selective marker. This method involves the transformation of plant tissue not containing meristematic parts i.e. internodal stem segments, leaves, tuber discs, flowers, pollen and/or roots and subjecting the plant cells or tissue with a T-DNA free of a selective marker or reporter-gene. Subsequently, plant cells go through a callus phase from which plants will develop. Subsequently, the plant tissue is assayed using a non-selective assay, such as PCR, enzyme assays or ELISA's. Assay-positive plants that are recovered with this method are non-chimeric and do not have to go through time-consuming crossing steps. This method has proven to be very useful.

WO 05/004585 describes a method for obtaining marker-free plants comprising a recombinant nucleic acid comprising a so-called P-DNA construct without a selective marker together with a construct with a selective antibiotic marker. By a positive selection for temporary marker gene expression linked with a negative selection for marker integration, plant cells are identified containing the P-DNA insertion, but lacking any copies of the marker gene. However, there is still a need for further alternative transformation methods.

The present invention further provides an alternative selection method in plant transformation processes.

Besides the fact that the presence of antibiotic resistance genes and other selective markers sequences in the final transgenic plant obtained is in most cases considered undesirable, there is a more general reluctance to the presence of non-plant sequences (i.e. trans sequences) in a transgenic plant. Many of the commercially available transgenic crops contain foreign regulatory elements such as the 35S promoter of the cauliflower mosaic virus and the terminator of the Agrobacterium nopaline synthase gene. Rommens et al. (2004) explains that transgenic plants approved for commercialisation contain on average eight genetic elements derived from viruses, bacteria or plants that are not sexually compatible with the target crop i.e. could not have been introgressed through traditional plant breeding. The invention therefore provides a plant that has been provided with additional nucleic acid sequences but which genetically modified plant essentially consists of cis plant sequences, for example a genetically modified potato plant that has been provided with additional (essentially) potato plant (coding) sequences from another potato variety, breeding clones or crossable species. The cis plant sequences are in their own native genomic context i.e. under control of their own promoter, having introns and their own trancription termination signal. Such a transgenic plant is herein further referred to as a cisgene plant. Alternatively, the embodiment describes a method for producing plants containing genetic elements derived from multiple genes from within the sexual compatibility group. A plant with such genetic elements is called an intragenic plant (Rommens et al., TIPS 2007). In a preferred embodiment, commercial interesting potato plants (such as Desiree or Bintje) have been provided with nucleic acid sequences isolated from other potato species (for example from a wild species of potato such as S. bulbocastanum).

Preferably, an obtained genetically modified Solanum plant comprises Solanum 5′ and 3′ sequences to direct transcription of a cDNA sequence or a genomic sequence. The used cDNA or genomic sequence may be orientated sense or antisense in relation to the used 5′ and 3′ sequences. The 5′ and 3′ sequences can be originally linked to the used cDNA or genomic sequence or they can normally be linked to another coding sequence in Solanum. In a preferred embodiment, the used coding sequence is a genomic sequence (in sense or antisense orientation) and the used 5′ and 3′ sequences are the ones that are linked to the genomic sequence under normal conditions (i.e. the 5′ and 3′ sequences are in operable linkage with their natural coding sequence).

The invention provides a method for obtaining a plant that has been provided with a nucleic acid of interest comprising providing:

a recombinant nucleic acid comprising said nucleic acid of interest transferring said recombinant nucleic acid to a plant cell producing a plant from said cell
and determining the presence of said nucleic acid of interest and the absence of vector backbone and/or border sequences,
wherein said nucleic acid of interest (or the to be transferred DNA, for example T-DNA) is essentially a plant nucleic acid.

For production of transformants free of vector DNA sequences it will be desirable to deliver DNA to cells that does not contain DNA sequences necessary for maintenance of the plasmid vector in the bacterial host, e.g., E. coli, such as antibiotic resistance genes, including but not limited to ampicillin, kanamycin, and tetracycline resistance, and prokaryotic origins of DNA replication (In such case, a DNA fragment containing the transforming DNA may be purified prior to transformation. Purification can be achieved by for example gel electrophoresis on an agarose gel, followed by recovery of a DNA fragment from the agarose gel.

By testing whether vector or vector backbone or border sequences are present, the invention provides a method for determining whether a plant has been provided with a recombinant nucleic acid that comprises a nucleic acid of interest, comprising optionally further providing said recombinant nucleic acid with a functional R-gene, transferring said recombinant nucleic acid to a plant cell, producing a plant from said cell, and exposing the resulting plant to at least one elicitor of Phytophthora and determining the presence or absence of a reaction to said at least one elicitor, further comprising determining the presence or absence of non-Solanum nucleic acid sequences. In a preferred embodiment, said non-Solanum nucleic acid sequences are non-Solanum T-DNA border sequences, i.e. in a preferred embodiment one tests for the presence or absence of (T-DNA) border sequences, especially in the cases in which at least one non-Solanum T-DNA border sequence is used.

The to be transferred nucleic acid sequence is free of nucleic acid sequences encoding a selective marker or a reporter gene, except that cisgenic marker or reporter sequences may be present. However, said method does not essentially need selection through treatment with a selective agent. Instead, selection for the desired transformant is achieved by testing for the presence of the nucleic acid of interest, for example by using commonly known nucleic acid techniques, such as detection by Polymerase Chain Reaction or by hybridisation with complementary sequences in routine Southern blotting experiments. It will also be apparent to those skilled in the art that the presence of a desired heterologous gene or gene fragment can be assayed by monitoring the presence or the absence or change in amount of the expression product of the gene. For example, an expressed protein allowing detection by ELISA (enzyme-linked immunosorbent assay) the presence of such a protein can be assayed by ELISA. Furthermore, the method provided herein may involve a bioassay or a chemical analytical method such as gas chromatography/mass spectometry (GC/MS).

Details of such a transformation and screening method are described in our co-pending application WO03/010319.

The phrase “said nucleic acid of interest is essentially a plant nucleic acid” is herein defined to refer to sequences obtained/derived/isolated from a plant (preferably a Solanum) i.e. the to be transferred nucleic acid sequence is essentially a Solanum nucleic acid sequence. If for example the Agrobacterium/binary vector method is selected as the transfer method, the sequences present on the T-DNA (i.e. the part that will be transferred) is essentially a Solanaceae nucleic acid sequence. The border sequences as well as the backbone of the used binary vector may comprise non-Solanaceae nucleic acid sequences. In a preferred embodiment, the used plant cell is a Solanaceae. This results in the production of a so-called cis(genic) plant, i.e. a plant that has been obtained through genetic modification by using species-own sequences or sequences from crossable species. For example a commercial potato variety that has been provided with a nucleic acid sequence isolated from a wildtype potato variety or from a crossable species. In a preferred embodiment, the cloning of the used sequences on the T-DNA is performed such that only Solanum sequences are present on the T-DNA, for example by using linkers that are derived from a Solanum to separate different coding sequences on the T-DNA. It is also possible to use a (modified) multiple cloning site that after cloning only results in the presence of a nucleotide sequence which is already present in the genomic background of the used plant cell. The term “essentially” is used herein to refer to the fact that although all sequences are in at least one (preferably non-genetically modified) plant (preferably a Solanum) naturally present, the genetic surroundings or setting may be somewhat different.

In a preferred embodiment, the invention provides a method for obtaining a plant that has been provided with a nucleic acid of interest comprising providing

a recombinant nucleic acid comprising said nucleic acid of interest transferring said recombinant nucleic acid to a plant cell producing a plant from said cell
and determining the presence of said nucleic acid of interest and the absence of vector backbone and/or border sequences,
wherein said nucleic acid of interest is essentially a plant nucleic acid,
wherein said plant nucleic acid is a Solanaceae nucleic acid. Even more preferred the used plant cell is a Solanaceae cell.

In yet another preferred embodiment, the invention provides a method for obtaining a plant that has been provided with a nucleic acid of interest comprising providing

a recombinant nucleic acid comprising said nucleic acid of interest transferring said recombinant nucleic acid to a plant cell producing a plant from said cell
and determining the presence of said nucleic acid of interest and the absence of vector backbone and/or border sequences,
wherein said nucleic acid of interest is essentially a plant nucleic acid,
wherein said nucleic acid of interest is a cDNA sequence or wherein said nucleic acid of interest is an inverted (repeat) sequence or wherein said nucleic acid of interest is a genomic sequences.

In a more preferred embodiment, said plant nucleic acid is an open reading frame that is under control of natural/native 5′ (promoter) and 3′ (terminator) nucleic acid sequences and in an even more preferred embodiment, said nucleic acid sequences is a genomic sequence. Use of this kind of sequences in combination with a Solanaceae plant cell results in the production of a so-called cisgenic plant, i.e. a plant that has been genetically modified by using species own sequences.

A further preferred embodiment is a method wherein said nucleic acid of interest is an R-gene. This way the invention provides for example cisgenic potato plants which are at least partial resistant against a pathogen (preferably P. infestans). Such plants are considered to be more easily excepted by ethic committees as well as by consumer groups and such plants at the same time reduce the use of for example fungicides.

The here-described method may further be complemented by determining the presence or absence of non-Solanum nucleic acid sequences. This confirms the absence of any non-plant nucleic acid sequences. Preferably said non-Solanum nucleic acid sequences are non-Solanum T-DNA border sequences.

The nucleic acid of interest may be present in the plant cell as an extra-chromosmally (episomal) replicating molecule but more preferably a method according to the invention results in integration of the recombinant nucleic acid (the to be transferred nucleic acid sequence) into the genome of said plant.

The invention further provides a plant obtainable by the method for obtaining a plant that has been provided with a nucleic acid of interest comprising providing

a recombinant nucleic acid comprising said nucleic acid of interest transferring said recombinant nucleic acid to a plant cell producing a plant from said cell
and determining the presence of said nucleic acid of interest and the absence of vector backbone and/or border sequences,
wherein said nucleic acid of interest is essentially a plant nucleic acid.

The invention also provides a genetically modified plant that has been provided with a cisgene, i.e. a gene obtained/derived/isolated from a same species but different variety. Preferably, said cisgene is under control of native 5′ (promoter) and 3′ (terminator) sequences and even more preferably said cis gene is a genomic sequence. Said genomic sequence can contain intron and exon sequences.

In a preferred embodiment, the obtained plant essentially only comprises nucleic acid sequences that are obtained from a plant and even more preferred from Solanum. Therefore the invention provides a method for determining whether a plant has been provided with a recombinant nucleic acid that comprises a nucleic acid of interest, comprising optionally further providing said recombinant nucleic acid with a functional R-gene, transferring said recombinant nucleic acid to a plant cell, producing a plant from said cell, and exposing the resulting plant to at least one elicitor of Phytophthora and determining the presence or absence of a reaction to said at least one elicitor, wherein said recombinant nucleic acid essentially consists of Solanum nucleic acid sequences, i.e. the to be transferred nucleic acid sequence is essentially a Solanum nucleic acid sequence. If for example the Agrobacterium/binary vector method is selected as the transfer method, the sequences present on the T-DNA (i.e. the part that will be transferred) is essentially a Solanaceae nucleic acid sequence. The border sequences as well as the backbone of the used binary vector may comprise non-Solanaceae nucleic acid sequences. Such transformants of Solanaceae will be selected which do not contain these border sequences and backbone sequences. This results in the production of a so-called cisgenic plant, i.e. a plant that has been obtained through genetic modification by using species-own sequences or sequences from crossable species, for example a commercial potato variety that has been provided with a nucleic acid sequence isolated from a wildtype potato species or a different potato variety or from a crossable species. In a preferred embodiment, the cloning of the used sequences on the T-DNA is performed such that only Solanum sequences are present on the T-DNA, for example by using linkers that are derived from or designed based on a Solanum genome to separate different coding sequences on the T-DNA. It is also possible to use a (modified) multiple cloning site that after cloning only results in the presence of a nucleotide sequence which is already present in the genomic background of the used plant cell. The term “essentially” is used herein to refer to the fact that although all sequences are in at least one (preferably non-genetically modified) plant (preferably a Solanum) naturally present, the genetic surroundings or setting may be somewhat different.

Further, the invention provides a method for determining whether a plant has been provided with a recombinant nucleic acid that comprises a nucleic acid of interest, comprising further providing said recombinant nucleic acid with a functional cisgene R-gene, transferring said recombinant nucleic acid to a plant cell, producing a plant from said cell, and exposing the resulting plant to at least one elicitor of Phytophthora and determining the presence or absence of a reaction to said at least one elicitor.

Such a method is very suitable for determining whether a nucleic acid of interest has been successfully transferred to a plant, i.e. whether plant transformation has succeeded.

The term “recombinant nucleic acid” is typically used to describe a double or single stranded DNA or RNA molecule and includes circular as well as linear forms of nucleic acid. In some of the embodiments, the recombinant nucleic acid used in a method of the invention at least comprises (i) a recombinant nucleic acid of interest and (ii) a functional R-gene. However the nucleic acid of interest and the functional R-gene may also be one and the same nucleic acid and hence in such an embodiment, the recombinant nucleic acid used in a method of the invention at least comprises a functional R-gene. In the latter case, said functional R-gene is used as a selection tool as well as to provide (at least partial) protection against a fungal infection/pathogen.

The recombinant nucleic acid used in a method of the invention may also comprise other sequences (i.e. besides a functional R-gene or the combination of a recombinant nucleic acid of interest and a functional R-gene like backbone/vector sequences as well as at least one and preferably two (optionally modified) T-DNA border sequences in the case of Agrobacterium mediated transformation. The T-DNA borders are derived from Agrobacterium and therefore plants containing these borders do not fall under the definition of a cisgenic or intragenic plants. The invention of WO05/004585 has been to search for functional plant T-DNA borders. The presence of these analogs was identified in Arabidopsis, rice, potato etc. (Rommens et al. Plant Physiol. 2004).

It is known in the art that deletion of these T-DNA borders often occurs during the integration of the T-DNA into the genome. We have analyzed five transformants obtained with construct pKGBA50mf-IR1.1 (de Vetten et al., 2003) in detail, and observed deletions of the T-DNA sequence from 39-237 bp at the RB side and from 79-1054 by at the LB side. Zhu et al. (2006) reported similar results. They analyzed 171 left borders and 134 right borders from independent transgenic rice plants and observed deletions of the T-DNA up to 35 bp at the RB side and up to 340 bp at the LB side. They stated that probably some transformants had deletions longer than 611 by at the LB side. Windels et al. (2003) analyzed 67 T-DNA/plant DNA junctions from Arabidopsis transformants. They observed deletions of T-DNA sequences up to 629 bp at the RB side and up to 2309 bp at the LB side. Thomas and Jones (2007) characterised 98 T-DNA/plant DNA junction sequences. 41 out of 42 LB junctions analysed did not retain sequences derived from the LB repeat. 36 out 56 RB junctions analysed did not retain sequences derived from the RB repeat.

On the basis of these observations it is expected that a vast majority of transformants can be selected that contain only Solanaceae sequence integrated in the genome.

For obtaining a maximum number of transformants with (deleted) non-integrated borders, said borders are preferably short with a minimum of 25 by left and right, however the use of longer borders is also feasible. As disclosed herein within the experimental part, a preferred left border setting is one in which an attenuation region in combination with a double left border is used. This setting prevents more frequent backbone integration (read through of the left border). Similar methods to prevent read-through are exemplified in EP-A-1 009 842. Integration of backbone vector (non-T-DNA) sequences in the plant genome frequently occurs during Agrobacterium tumefaciens transformation (Kononov et al. 1997; Ramanathan and Veluthambi, 1995; Van der Graaff et al.; 1996; Wenck et al., 1997; Wolters et al., 1998). Wang et al. (1987) reported that “flanking sequences of the border repeats enhance (on the right) or attenuate (on the left) their activity”. They defined an ‘attenuation region’: a 363-bp AT-rich EcoRI/BclI fragment of the T-DNA near the LB of plasmid pTiC58. De Buck et al. (2000) stated that “it is possible that deletion of the inner border region, a piece of T-DNA present in the original Ti plasmid from which the vector was derived, causes inefficient nicking of the LB repeat, which results in read-through past the LB and the transfer of downstream-located vector sequences”. Kuraya et al. (2004) reported that “transfer of the ‘vector backbone’ from the control vectors resulted mainly from inefficient termination of formation of the transfer intermediate of the T-DNA, and additional LB sequences effectively suppressed such transfer”.

On the basis of these reports we have cloned in one of our examples an extra nopaline type LB together with the flanking attenuation region outside the LB of a binary vector, to prevent integration of backbone vector DNA in the plant genome.

As described above the recombinant nucleic acid used in a method of the invention may further comprise vector sequences if they are cisgenic, i.e. obtained from the same plant or the same sexual compatibility group. The vector sequences may for example comprise a selection marker for maintaining the recombinant nucleic acid in E. coli and/or in Agrobacterium.

It is clear to the skilled person that if one would like to introduce multiple nucleic acids of interests, the recombinant nucleic acid used in a method of the invention may comprise multiple nucleic acids of interest.

Other sequences present on the recombinant nucleic acid used in a method of the invention are promoter and/or terminator sequences, preferably functionally linked/coupled to a nucleic acid of interest and/or to an R-gene. Preferred embodiments of these promoter and/or terminator sequences are described later on.

It is clear to the skilled person that a vast amount of nucleic acids of interest can be thought of and which are all included herein. In one embodiment, said nucleic acid of interest allows for regulation of the expression of a target gene in said genome. Both up-regulation (or over-expression) and down-regulation can be achieved by “sense” technology. If a full-length copy of the target gene is inserted into the genome a range of phenotypes can be obtained, some over-expressing the target gene, some under-expressing. A population of plants produced by this method may then be screened and individual phenotypes isolated. A preferred embodiment comprises a method wherein said regulation comprises down-regulation. The inhibition of expression of a target gene, commonly referred to as “gene silencing” can be achieved by “antisense down-regulation” and “sense down-regulation” (also, referred to as “co-suppression”). In antisense down-regulation, a DNA fragment which is complementary to all or part of an endogenous target gene is inserted into the genome in reverse orientation. While the mechanism has not been fully elucidated, one theory is that transcription of such an antisense gene produces mRNA which is complementary in sequence to the mRNA product transcribed from the endogenous gene. The antisense mRNA then binds with the naturally produced “sense” mRNA to form a duplex which inhibits translation of the natural mRNA to protein. Antisense down-regulation technology is well-established in the art and used routinely in laboratories around the world. Gene silencing can therefore be achieved by inserting into the genome of a target organism a copy of at least a fragment of the target gene coding sequence which copy may comprise either the whole or part or be a truncated sequence and may be in sense or antisense orientation. Additionally, intron sequences which can be obtained from the genomic gene sequence may be used in the construction of suppression vectors. There have also been reports of gene silencing being achieved within organisms of both the transgene and the endogenous gene where the only sequence identity is within the promoter regions.

In a much preferred embodiment, in the invention a recombinant nucleic acid is used, wherein said nucleic acid of interest includes an inverted repeat of at least part of a polynucleotide region of said target gene. Although antisense and sense down-regulation can result in complete silencing of the target gene, the efficiency is generally not very high. A maximum of 25% of the antisense transformants display complete silencing, while only about 10% of the transformants obtained with sense constructs show some level of silencing (Smith et al. 2000 Nature 407: 319-320; Wolters and Visser, 2000 Plant Mol Biol 43: 377-386). Recently, it has been observed that the inhibition of a selected target gene within an organism is enhanced, when the gene silencing vector includes an inverted repeat of all or part of a polynucleotide region of the target gene. The inverted repeat sequence may consist of for example a T-DNA with one promoter driving expression of a sense copy of the cDNA together with another promoter in front of an antisense copy of the same cDNA (Chuang and Meyerowitz, 2000 Proc Natl Acad Sci USA 97: 4985-4990) or the T-DNA may contain a cDNA sequence flanked on both ends by a promoter (LaCount et al. 2000 Mol Biochem Parasit 111: 67-76), or the T-DNA may contain a promoter driving transcription of an inverted repeat of (part of) the cDNA (Hamilton et al. 1998; Smith et al. 2000 Nature 407: 319-320; Wang and Waterhouse, 2000 Wang M B, Waterhouse P M (2000) Plant Mol Biol 43: 67-82) or the promoter (Mette et al. 2000 EMBO J 19: 5194-5201) of the gene to be silenced. Some inverted repeat constructs are reported to result in 100% of the transformants displaying silencing of the target gene (Smith et al. 2000 Nature 407: 319-320) in case of using an intron as a spacer between the repeats. The spacer fragment contributes to the stability of the perfect inverted repeat sequences, but is not required for the specificity of the silencing.

In a particularly preferred embodiment, a nucleic acid of interest encodes a granule-bound starch synthase (GBSSI) enzyme or comprises an antisense sequence. The inclusion of a short repeated region of the granule-bound starch synthase (GBSSI) enzyme of potato within a transgene results in a striking increase in the frequency of completely silenced transformants.

Also provided is a nucleic acid of interest that allows for expression of a heterologous, cisgenic polypeptide in a plant cell. Suitable polypeptides are manifold, typical examples of foreign nucleic acid or genes that are of interest to transfer into plants are those encoding for proteins and enzymes that modify metabolic and catabolic processes. Other examples are genes that may encode a protein giving added nutritional value to the plant as a food or crop. Typical examples include plant proteins that can inhibit the formation of anti-nutritive factors and plant proteins that have a more desirable amino acid composition (e.g. a higher lysine content than the non-transgenic plant). In a preferred embodiment, said nucleic acid of interest encodes a polypeptide which comprises an enzyme. The nucleic acid of interest may also code for an enzyme that can be used in food processing such as chymosin, thaumatin and alpha-galactosidase. The nucleic acid of interest may also code for an agent for introducing or increasing pathogen resistance. Preferably, the gene of interest is a gene encoding for a protein or peptide having a high nutritional value, a feedback-insensitive amino acid biosynthesis enzyme such as dihydrodipicolinate synthase (EC 4.2.1.52, DHPS), an enzyme or peptide conferring disease resistance, a sense or antisense transcript such as that for patatin, ADP-glucose pyrophosphorylase, alpha-amylase, branching enzyme, granule-bound starch synthase, soluble starch synthases, a protease or a glucanase.

In yet another embodiment, a nucleic acid of interest is a nucleic acid sequence that modulates the texture properties of plant tissues and organs, especially after heat treatment, such as cooking. Preferably, the cooking type of potato tubers can be altered, from example from “mealy” to “firm” or the other way around. An example of such a gene is a sttlrp nucleic acid that encodes a so-called StTLRP protein (Solanum tuberosum tyrosine and lysine rich protein). FIG. 3 provides the nucleic acid sequence of such a sttlrp nucleic acid.

StTLRP proteins (and sttlrp nucleic acids encoding these) are suitable for modulating texture characteristics of plant cell walls, especially cells (and tissues consisting largely thereof) which lack a rigid (lignified) secondary cell wall. A significant correlation between mRNA expression levels and firmness of potato tubers was found. Potato genoptypes comprising a specific allele of the sttlrp gene (the sttlrp Δ7 allele) had sttlrp mRNA levels which were much higher (e.g. 64-fold upregulated) than genotypes lacking this allele. These genotypes had on average much firmer tissue after cooking. In contrast, genotypes which lacked the Δ7 allele were mealy after cooking. Therefore, particular high expressing alleles are able to confer a ‘firm’, ‘non-mealy’ phenotype to the potato tubers, while low expressing alleles (and thus the absence of high expressing alleles) are capable of conferring a ‘mealy’ phenotype. The genomic and cDNA nucleic acid sequences of the Δ7 allele are shown in FIG. 3.

In yet another preferred embodiment, the invention provides a method wherein the nucleic acid of interest is also a functional R-gene. The term R-gene is typically used to describe a DNA sequence that encodes a gene product that is capable of providing a plant with (at least partial) resistance against a pathogen. Even more preferred, the R-gene used in the selection process is also used to provide the obtained plant with resistance. Preferably, the R-gene used in the selection process and the further functional R-gene (as a nucleic acid of interest) are chosen such that they provide complementary (and even more preferably partially overlapping) protection against a pathogen. The use of at least two preferably different R-genes results in so-called stacking of R-genes.

Whether or not multiple, different R-genes provide a complementary and/or overlapping effect is for example tested by ATTA, i.e. Agrobacterium tumefaciens transient expression assay. In this assay the nucleotide sequence coding for an R-gene which is to be tested is introduced into an Agrobacterium strain which is also used in protocols for stable transformation. After incubation of the bacteria with acetosyringon or any other phenolic compound which is known to enhance Agrobacterium T-DNA transfer, a certain amount of the Agrobacterium culture is infiltrated into an in situ plant leaf (for example a tobacco or potato or tomato plant) by injection after which the plants are placed in a greenhouse and infected with a pathogen (for example P. infestans). After 2-5 days the leaves can be scored for occurrence of resistance symptoms. Multiple different R-genes can be tested versus multiple different P. infestans isolates or versus multiple different elicitors.

Another method to test whether or not multiple, different R-genes provide a complementary and/or overlapping effect is by using a detached leaf assay with, preferably well-characterised, isolates (for example P. infestans isolates) or with an effector (such as an elicitor) which disclose that iterative functional allele mining in Solanum and Phytophthora is a powerful tool to identify and characterize R-avr combinations at a much higher discriminatory resolution than was previously possible using only isolate screens.

Wild Solanum species have previously been subject to mapping of genes for resistance to P. infestans. A summary of mapped R-genes for foliage late blight resistance is given in Table 1. Currently 4 R-genes for late blight resistance have been cloned and all belong to the NB-LRR class of plant R-genes; R1 and R3α from S. demissum (Ballvora et al., 2002; Huang et al., 2005) and Rpi-blb1 and Rpi-blb2 from S. bulbocastanum (van der Vossen et al, 2003, 2005). Examples of R-genes that can be used as a nucleic acid of interest are provided in Table 1.

However, also R-genes isolated from other plants than potato can be used in a method of the present invention. Preferably the used functional R-gene and the elicitor are matched. For example, done wants to use Rps1b from soybean plants, the used elicitor is preferably an elicitor of Phytophthora sojae (preferably Avr1b-1). Other examples are provided in the Table below.

| | | Avr | | |
|---|---|---|---|---|
| Host | R gene | gene | Pathogen | |
| Potato | R3a | Avr3a | Phytophthora infestans | Oomycete |
| Potato | Rpi- | ipiO | Phytophthora infestans | Oomycete |
| | blb1 | | | |
| Soybean | Rps1b | Avr1b | Phytophthora sojae | Oomycete |
| Arabidopsis | Rpp1 | Atr1 | Hyaloperonospora | Oomycete |
| thaliana | | | parasitica | |
| Tomato | Cf2, | Avr2, | Cladosporium fulvum | Fungus |
| | Cf4, | Avr4, | | |
| | Cf9 | Avr9 | | |
| Tomato | Pto | AvrPto | Pseudomonas syringae | Bacteria |
| | | | pv. tomato | |

Typically, the term “functional (R-) gene” is used herein to refer to a gene that is transcribable, i.e. the gene product is expressed (and provides a product that is capable of interacting with at least one Phytophthora elicitor). Suitable genes are provided in Table 1.

There are multiple ways in which the recombinant nucleic acid can be transferred to a plant cell, for example Agrobacterium mediated transformation. However, besides by Agrobacterium infection, there are other means to effectively deliver DNA to recipient plant cells when one wishes to practice the invention. Suitable methods for delivering DNA to plant cells are believed to include virtually any method by which DNA can be introduced into a cell, such as by direct delivery of DNA such as by PEG-mediated transformation of protoplasts (Omirulleh et al., 1993), by desiccation/inhibition-mediated DNA uptake (Potrykus et al., Mol. Gen. Genet., 199:183-188, 1985), by electroporation (U.S. Pat. No. 5,384,253), by agitation with silicon carbide fibers (Kaeppler et al., 1990; U.S. Pat. No. 5,302,523; and U.S. Pat. No. 5,464,765), and by acceleration of DNA coated particles (U.S. Pat. No. 5,550,318; U.S. Pat. No. 5,538,877; and U.S. Pat. No. 5,538,880). Through the application of techniques such as these, certain cells from virtually any plant species may be stably transformed, and these cells developed into transgenic plants.

In case Agrobacterium mediated transfer is used, it is preferred to use a substantially virulent Agrobacterium such as A. tumefaciens, as exemplified by strain A281 or a strain derived thereof or another virulent strain available in the art. These Agrobacterium strains carry a DNA region originating from the virulence region of the Ti plasmid pTiBo542 containing the virB, virC and virG genes. The virulence (vir) gene products of A. tumefaciens coordinate the processing of the T-DNA and its transfer into plant cells. Vir gene expression is controlled by virA and virG, whereby virA upon perception of an inducing signal activates virG by phosphorylation. VirG, in turn, induces the expression of virB,C,D,E. These genes code for proteins involved in the transfer of DNA. The enhanced virulence of pTiBo542 is thought to be caused by a hypervirulent virG gene on this Ti plasmid (Chen et al. Mol. Gen. Genet 230: 302-309, 1991).

In Microprojectile Bombardment, particles may be coated with nucleic acids and delivered into cells by a propelling force. Exemplary particles include those comprised of tungsten, gold, platinum, and the like. It is contemplated that in some instances DNA precipitation onto metal particles would not be necessary for DNA delivery to a recipient cell using microprojectile bombardment. However, it is contemplated that particles may contain DNA rather than be coated with DNA. For microprojectile bombardment transformation in accordance with the current invention, both physical and biological parameters may be optimized. Physical factors are those that involve manipulating the DNA/microprojectile precipitate or those that affect the flight and velocity of either the macro- or microprojectiles. Biological factors include all steps involved in manipulation of cells before and immediately after bombardment, such as the osmotic adjustment of target cells to help alleviate the trauma associated with bombardment, the orientation of a target tissue relative to the particle trajectory, and also the nature of the transforming DNA, such as linearized DNA or intact supercoiled plasmids. It is believed that pre-bombardment manipulations are especially important for successful transformation. In the preferred embodiment target tissue will be transformed with linearized DNA not containing backbone plasmid DNA sequences.

Accordingly, it is contemplated that one may wish to adjust various bombardment parameters in small-scale studies to fully optimize the conditions. One may particularly wish to adjust physical parameters such as DNA concentration, gap distance, flight distance, tissue distance, and helium pressure. It is further contemplated that the grade of helium may effect transformation efficiency. One also may optimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation.

The transforming DNA used for microprojectile transformation may contain an expression cassette comprising generally of a cDNA, gene or genes which one desires to introduce into the cells, and still further, may include a promoter and 3′ region operatively linked to the heterologous gene. The optimized gene construct is described in present invention. The DNA segment may additionally include a second, third, fourth, fifth, sixth, or any additional number of heterologous genes capable of being placed on a single DNA molecule and transformed into a recipient cell.

The recipient plant cells for transformation with the recombinant nucleic acid in a method according to the invention may be from potentially any transformable monocot or dicot plant. Preferred monocot plant cells for use with the invention are from rice, wheat, barley, oats, rye, millet, sorghum, sugarcane, turfgrass and maize. Preferred dicot plant cells for use with the invention include cotton, tomato, citrus, tobacco, soybean and particularly potato and cassava.

After transfer of the recombinant nucleic acid a plant is produced from said genetically modified plant cell. This comprises regeneration methods that are well known in the art and need no further elaborate discussion.

If the resulting plant comprises an R-gene which provides resistance against Phytophthora, it may be subjected at any age to at least one strain or elicitor of Phytophthora. In principle a resulting plant with only one leaf can already be subjected to at least one strain or elicitor of Phytophthora. Moreover, plants can be tested in vitro as well as in vivo. A preferred embodiment, involves testing two weeks after potting from an in vitro culture.

An important advantage of a method according to the invention is, next to the fact that the marker gene is a cisgene, the fact that the obtained plants need not be grown in a selective medium, e.g. a medium comprising a herbicide, antibiotic, amino acid analog or the like. Moreover, a method of the invention does not comprise the use of additional equipment which is for example needed in case a GFP or GUS marker is used.

As described above also functional R-genes of plants other than potato can be used in a method of the invention, for example when transforming soya with the Rps1b gene from soybean or when transforming tomato a Cf gene from tomato can be used.

The invention therefore also provides a method for determining whether a plant has been provided with a recombinant nucleic acid that comprises a nucleic acid of interest, comprising further providing said recombinant nucleic acid with a functional R-gene, transferring said recombinant nucleic acid to a plant cell, producing a plant from said cell, and exposing the resulting plant to at least one elicitor of for example Cladosporium and determining the presence or absence of a reaction to said at least one elicitor. The used plant cell is in this particular example a Lycopersicon cell.

A method of the invention optionally further comprises testing of copy number.

Such an analysis is preferably performed on DNA/RNA level. This includes the isolation of nucleic acid from plantlets or a pool of plantlets according to standard methodologies (Sambrook et al., 1989). The nucleic acid may be genomic DNA, RNA or mRNA. Also rearrangements can be detected. The invention provides the use of a mRNA detection method for determining whether a nucleic acid construct in a transformed plant cell or progeny thereof is sufficiently integrated into a plant genome to be transcribed into a mRNA construct. The specific nucleic acid of interest, being part of the transgene is identified in the sample directly (DNA) or indirectly (RNA) using amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals. A variety of different assays are contemplated in the screening of transgenic plants created using the methods of the current invention. These techniques can be used to detect for both the presence of particular genes as well as rearrangements that may have occurred in the gene construct. The techniques include but are not limited to, polymerase chain reaction (PCR), fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient gel electrophoresis, restriction fragment length polymorphism (RFLP) and PCR-SSCP or chip-based DNA technologies. The invention provides the use of a nucleic acid detection method for determining whether a transformed plant cell or progeny thereof is transformed with a recombinant nucleic acid comprising a T-DNA construct or a functionally equivalent nucleic acid construct allowing integration into a genome of a plant cell. Furthermore, the invention provides the use of a nucleic acid detection method for determining whether a transformed plant cell or progeny thereof is transformed with a recombinant nucleic acid comprising testing said cell or said progeny for the presence or absence of undesired vector material such as vector backbone sequences. For example, a method according to the invention can be used to check whether a transformed plant cell is essentially free of ancillary unwanted nucleic acids. The transformed plantlet, identified by nucleic acid detection methods, will then be allowed to mature into plants. After the regenerating plants have reached the stage of shoot and root development, they may be transferred to a greenhouse for further growth and testing.

Control of the copy number of introduced transgenes and an efficiently production of low-copy transformants can, especially in case for transformation via gun bombardment, be achieved by end-modification of nucleic acid segments by dephosphorylation or by blunting the ends of the nucleic acid segments (WO99/32642).

Preferably, the used functional R-gene and/or the used nucleic acid of interest are obtained/isolated from a Solanaceae including its own 5′ (promoter) and 3′ (terminator) sequences, i.e. the used functional R-gene and/or the used nucleic acid of interest are preferably under regulation of their natural/native promoter and terminator region.

In a preferred embodiment, the invention provides a method for determining whether a plant has been provided with a recombinant nucleic acid that comprises a nucleic acid of interest, comprising further providing said recombinant nucleic acid with a functional R-gene, transferring said recombinant nucleic acid to a plant cell, producing a plant from said cell, and exposing the resulting plant to at least one elicitor of Phytophthora and determining the presence or absence of a reaction to said at least one elicitor, wherein said nucleic acid of interest and said functional R-gene are the same. Such a method does not rely on a selective marker gene and does thus not need selection through treatment with a selective agent. In this embodiment, the functional R-gene has a dual function, i.e. said R-gene is used to determine which plants have been provided with a recombinant nucleic acid and is also used to confer (at least partial) resistance against a pathogen.

In yet another embodiment, the invention provides a method for determining whether a plant has been provided with a recombinant nucleic acid that comprises a nucleic acid of interest, comprising further providing said recombinant nucleic acid with a functional R-gene, transferring said recombinant nucleic acid to a plant cell, producing a plant from said cell, and exposing the resulting plant to at least one elicitor of Phytophthora and determining the presence or absence of a reaction to said at least one elicitor, wherein said nucleic acid of interest is a functional R-gene. Examples of suitable R-genes are provided in Table 1. Such a method is actually a method to provide a plant with at least partial resistance against a pathogen. In this embodiment, the functional R-gene used to determine the presence or absence of a recombinant nucleic acid may also be used to confer resistance. In the latter case, the produced plant comprises at least two functional R-genes. It is clear to the skilled person that a plant can also be provided with three or four or five or six (preferably different) functional R-genes. Preferably the used R-genes are chosen such that they provide complementary protection against a pathogen. Even more preferably the obtained protection is a broad scope protection resulting in a (durable) resistant phenotype.

As described above multiple reactions can be used to determine whether a reaction to an elicitor is present. In a preferred embodiment, said reaction is a hypersensitive response (HR). Whether or not an HR is induced is for example tested with an agroinfection or agroinfiltration. Both techniques are outlined in more detail in the experimental part herein. If a representative Phytophthora strain is used instead of an (isolated) elicitor, the skilled person preferably uses inoculation.

The transferred nucleic acid may be present in the plant cell as an extra-chromosmally (episomal) replicating molecule but more preferably a method according to the invention results in integration of the recombinant nucleic acid into the genome of said plant.

In a preferred the invention provides a method for determining whether a plant has been provided with a recombinant nucleic acid that comprises a nucleic acid of interest, comprising further providing said recombinant nucleic acid with a functional R-gene, transferring said recombinant nucleic acid to a plant cell, producing a plant from said cell, and exposing the resulting plant to at least one elicitor of Phytophthora and determining the presence or absence of a reaction to said at least one elicitor, wherein said nucleic acid of interest and/or said functional R-gene is/are cDNA sequences. In another preferred embodiment, said nucleic acid of interest is an inverted (repeat) sequence. An example of an inverted repeat sequence is an inverted repeat sequence of GBSS.

In yet another preferred embodiment, the invention provides a method for determining whether a plant has been provided with a recombinant nucleic acid that comprises a nucleic acid of interest, comprising further providing said recombinant nucleic acid with a functional R-gene, transferring said recombinant nucleic acid to a plant cell, producing a plant from said cell, and exposing the resulting plant to at least one elicitor of Phytophthora and determining the presence or absence of a reaction to said at least one elicitor, wherein said nucleic acid of interest and said functional R-gene are genomic sequences, more preferably genomic Solanaceae sequences. As a result the genes are present within their normal/native exon/intron context. In a most preferred embodiment, said nucleic acid of interest and said functional R-gene are not only genomic sequences (more preferably genomic Solanaceae sequences) but are also controlled by their native/natural 5′ and 3′ sequences, i.e. by a promoter and a terminator sequence as present in the original/natural/native setting.

As already described, a method of the invention can be performed by using different functional R-gene/elicitor (or Phytophthora) combinations. In one of the embodiments the functional R-gene is Rpi-blb-1 or Rpi-sto1 or Rpi-pta1 and the used elicitor is RD7 (IPI-0) and hence the invention also provides a method for determining whether a plant has been provided with a recombinant nucleic acid that comprises a nucleic acid of interest, comprising further providing said recombinant nucleic acid with a functional R-gene, transferring said recombinant nucleic acid to a plant cell, producing a plant from said cell, and exposing the resulting plant to at least one elicitor of Phytophthora and determining the presence or absence of a reaction to said at least one elicitor, wherein said functional R-gene is a gene encoding Rpi-blb-1 or Rpi-sto1 or Rpi-pta1 or a gene encoding a functional fragment thereof or a gene encoding a derivative thereof. In an even more preferred embodiment, said elicitor is RD7 (IPI-0). As disclosed in more detail in one of our co-pending applications, Rpi-sto1 and Rpi-pta1 are homologues of Rpi-blb-1 and can interact with RD7 (IPI-0). FIG. 4 shows an alignment between Rpi-blb-1 and Rpi-sto1 and Rpi-pta1.

In yet another embodiment, the used functional R-gene is a gene encoding blb-3 as depicted in FIG. 2 and hence in another embodiment, the invention provides a method for determining whether a plant has been provided with a recombinant nucleic acid that comprises a nucleic acid of interest, comprising further providing said recombinant nucleic acid with a functional R-gene, transferring said recombinant nucleic acid to a plant cell, producing a plant from said cell, and exposing the resulting plant to at least one elicitor of Phytophthora and determining the presence or absence of a reaction to said at least one elicitor, wherein said functional R-gene is a gene encoding blb3 or a gene encoding a functional fragment thereof or a gene encoding a derivative thereof. In yet another embodiment, said functional R-gene is a gene encoding blb-3 as depicted in FIG. 2.

Blb3 is a LZ-NBS-LRR type of R-gene and as described in a co-pending application, provides resistance to a range of P. infestans isolates.

A functional fragment of a blb-3 nucleic acid sequence is a truncated (n-terminal, C-terminal, internally or a combination thereof) sequence of the complete blb-3 nucleic acid sequence. The truncated sequence has a comparable function and/or activity if compared to the full-length sequence, i.e. a functional fragment is capable of providing a member of the Solanaceae family with race non-specific (at least partial) resistance against an oomycete pathogen. Such a truncated sequence is for example tested in the herein already outlined ATTA methodology or by an effector screen with the matching effector (elicitor).

Examples of a derivative are allelic variants of blb-3.

The invention further provides a plant obtainable by a method of the invention. In a preferred embodiment, said plant is a commercially interesting potato variety such as Bintje, Desiree or Premiere, Spunta, Nicola, Favorit, Russet Burbank, Aveka or Lady Rosetta. The identity of plants of the invention is for example determined by performing a PCR to determine whether a functional R-gene is present and whether selection markers such as kanamycin are absent. The R-gene is preferably present in its non-natural background, e.g. Rpi-blb1 or 3 in a non S. bulbocastanum background or R3 in a non S. demissum background or Rpi-sto1 in a non S. stoloniferum background or Rpi-pta1 in a non S. patita background. The same is true for the used nucleic acid of interest e.g. if a gene of interest is isolated from S. microdontum, said gene of interest is preferably introduced in a non S. microdontum background. In other words, the to be introduced traits are preferably introduced in a variety different from the species from which they were cloned or isolated.

In yet another preferred embodiment, the herein used R-gene and/or nucleic acid of interest is foreign to the plant cell. The term “foreign” is herein used to describe the situation in which the R-gene and/or nucleic acid of interest is heterologous with respect to the host cell (i.e. derived from a cell or organism with a different genomic/genetic background if compared to the cell used for transferring) or the R-gene and/or nucleic acid of interest is homologous with respect to the used host cell but located in a different genomic environment than the naturally occurring counterpart of said R-gene and/or nucleic acid of interest (for example surrounded by different genes if compared to the natural gene layout).

As described above, the used R-gene and the used nucleic acid of interest are preferably controlled by their own 5′ (promoter) and 3′ (terminator) nucleic acid sequences. The presence of such 5′ (promoter) and 3′ (terminator) nucleic acid sequences may also be determined by for example PCR or sequence analysis. As is also described above, the used R-gene and the used nucleic acid of interest are genomic sequences, i.e. optionally intron and exon sequences are present. Again, the presence of such intron and/or exon sequences can also be determined by using PCR or sequence analysis.

Moreover, the plants of the invention are preferably, non-chimeric and/or marker-free and comprise no backbone sequences and comprise no border sequences. A plant obtained by a method of the invention does not need any recombination event (to for example excise a selection marker) and does also not need any crossing event (to for example cross out a selection marker), i.e. a plant obtainable by a method of the invention does not need any additional recombination events or sexual cycle. However, an obtained plant can be used as crossing parent for new varieties. The thus obtained plant has a genomic background which is essentially identical to the genetic background of the plant cell used for transformation and said obtained plant does also not contain any traces of recombination events.

The described methods can be applied to multiple types of plants. Preferred monocot plants (cells for use with the invention) are from rice, wheat, barley, oats, rye, millet, sorghum, sugarcane, turfgrass and maize. Preferred dicot plants (cells for use with the invention) include cotton, tomato, citrus, tobacco, soybean and particularly potato and cassava.

The invention will be explained in more detail in the following, non-limiting examples.

### EXPERIMENTAL PART

### Materials and Methods

### Agroinfection

Recombinant A. tumefaciens GV3101 strains carrying candidate effectors (obtained from OSU) in pGR106 were used to screen for a response in Solanum. The pGR106-CRN2 and the pGR106 empty vector (Jones et al., 1999; Takken et al., 2000; Torto et al., 2003) were used as a positive and negative control respectively. Cultures were grown for 2 days at 28° C. on solid agar LB medium supplemented with antibiotics. Excess of bacteria was inoculated by piercing the leaf at both sides of the mid-vein. Local and systemic symptoms were visually scored every 2-4 days. For mature plant inoculations, usually three leaves from three to four week old plants was used, and the leaf age was rotated for replicates of the various treatments.

### Application of Agroinfection in Solanum

Resistance to PVX is known to occur in Solanum and would interfere with large-scale screenings with the binary PVX vector. To determine the extent to which the assay is applicable to the diverse Solanum germplasm, we inoculated plants corresponding to 80 Solanum clones from 31 species with A. tumefaciens carrying pGR106 and pGR106-CRN2 (Vleeshouwers et al., 2006). Treatment with the pGR106-CRN2 strain consistently caused necrosis around the inoculation sites on 50 plant clones (63%) whereas the empty vector strain caused no symptoms. We concluded that these plants are suitable for PVX agroinfection assays. The remainder of the Solanum clones tested were not suitable for the assay. Responses to both the positive and negative controls occurred in 20 clones and were regarded as nonspecific reactions to PVX. Ten other clones showed no necrotic response to pGR106-CRN2 possibly because the gene insert was not expressed in planta. In summary, it appears that the PVX assay is suitable for about 80% of the species examined and 60% of the clones. Therefore, before each screening with a large set of candidate effectors from P. infestans, a pre-screening with pGR106-CRN2 and the pGR106 empty vector is performed.

### Agroinfiltration

Co-infiltrations of Agrobacterium strains carrying the R gene and the AVR gene were performed in N. benthamiana. Recombinant A. tumefaciens cultures were grown in LB medium (10 gram bacteriological peptone, 10 gram NaCl and 5 gram yeast extract in 1 liter MQ water) supplemented with 50 mg/L Rifamplicin and 50 mg/L Kanamycin for the LBA4404 constructs. The AGL-1 was supplemented with 5 mg/L tetracycline and 50 mg/L kanamycin (the empty AGL-1 was only grown on tetracycline). After one or two days a calculated amount of culture (according to OD 0.5 at 600 nm) was transferred to YEB medium (5 gram beef extract, 5 gram bacteriological peptone, 5 gram sucrose, 1 gram yeast extract, 2 ml 1 M MgSO₄ in 1 liter MQ water) supplemented with Kanamycin for all strains, but for AGL-1 empty vector tetracycline was used. After 1 day overnight cells were centrifuged at 3500 rpm and resuspended in MMA medium (20 gram sucrose, 5 gram MS salts and 1.95 gram MES) supplemented with 1 ml 200 mM acetosyringone to an final OD of 0.5. Both constructs (R-gene and PEX) were mixed 1:1 before infiltration into 4-6 weeks old plants with a 3 ml syringe. Symptom development was monitored after 5 to 6 days.

### Example 1

### Selection of Cisgene R3a Transformants

Construction of Vector pBINmf::R3a

pB1121-derived binary vector pPGB-1S (Kuipers et al, 1995) was digested with enzymes PmeI and ClaI to remove the NptII gene. The ClaI sticky end was made blunt-ended by Klenow polymerase treatment, after which the vector DNA was circularized by blunt-end ligation using T4 DNA ligase. This resulted in vector pPGBmf (marker-free) containing in the T-DNA the potato GBSSI promoter followed by the NOS terminator as an 1140-bp HindIII/EcoRI fragment. Construct pPGBmf was digested with HindIII and EcoRI, resulting in two fragments of 1140 by and 9681 bp. The 9681-bp fragment containing the inner LB (left border) sequence, LB, backbone vector DNA, RB and inner RB sequence was isolated from an agarose gel.

A double stranded oligo was made by annealing primers AWO1 (5′-AGCTTGGCGCGCCCGGGTTAATTAAG-3′ (SEQ ID NO: 2)) and AWO2 (5′-AATTCTTAATTAACCCGGGCGCGCCA-3′ (SEQ ID NO: 3)). This sequence contains HindIII and EcoRI sticky ends and restriction sites for AscI, SmaI and PacI. The oligo was ligated to the 9681-bp HindIII/EcoRI fragment of pPGBmf, resulting in vector pBINmf (9707 bp).

Vector pBINmf::R3a was developed by the cloning of a PacI/AscI genomic DNA fragment of SH23-2 into PacI/AscI-digested pBINmf. SH23-2 is a subclone of the Bacterial Artificial Chromosome (BAC) SH23 (Huang et al. 2005). This BAC was partially digested with Sau3AI and the 7-10 kb fraction was ligated into the BamHI site of vector pBINPLUS.

The T-DNA in vector pBINmf:R3a contains a 9-kb fragment of the SH23-2 pBINPLUS subclone, in which the Coding Sequence (CDS) of gene R3a is situated. The 3849-bp CDS is (after transformation of the plant) regulated by the original promoter and terminator of R3a which are both present on SH23-2. The genomic fragment SH23-2 is isolated from the diploid potato clone SH83-92-488 (Huang et al. 2005).

The binary vector pBINmf::R3a was transformed into Agrobacterium tumefaciens strain AGL0 or AGL-1 by triparental mating, using helper plasmid pRK2013 (Figurski and Helinski, 1979).

### Transformation of Potato and Selection of Transformants

Internodal cuttings from in vitro grown plants of potato cultivar ‘Desiree’ were used for transformation by Agrobacterium tumefaciens co-cultivation, according to the protocol described by Visser et al. (1991a).

Potato cultivar ‘Desiree’ was transformed with pBINmf::R3a in A. tumefaciens AGL-1. No selection was performed. After four weeks the first shoots were harvested and harvesting of shoots continued for about three months. No more than two regenerants per stem explant were harvested and shoots were allowed to grow on MS30 medium in a glass jars (diameter 10 cm) or plastic jars (diameter 15 cm). Each jar contained 5 cuttings. A 2 to 3 cm space was left between the cuttings and the inner wall of the jars.

In Vitro Selection with avr3

In vitro plantlets were grown at 24° C. and 16 h light/8 h dark. Plantlets with 3 to 4 fully developed leaves were used for in vitro inoculation. Depending on the potato genotype, after 2 to 4 weeks plantlet were ready for inoculation.

The 3 largest leaves of each in vitro plantlet were inoculated (1 spot per leaf) by pipetting 10 μl droplets of a zoospore suspension of 2.5×10⁴ spores/ml on the adaxial side. Inoculum preparation and inoculation were performed in sterilized conditions. Leaves touching the inner wall or the lid of the jars or the medium were excluded because we found these leaves to be more susceptible than others of the same plants. The jars with inoculated in vitro plantlets were incubated in the same climate chamber as the trays with inoculated detached leaves.

Non-transformed plantlet showing a susceptible interactions were scored as S (spreading lesion with massive sporulation) or class SQ (spreading lesion with no or little sporulation). Putative transformants showing a resistant interactions were scored as class R (no symptom or localized HR-like necrosis) or class RQ (trailing HR necrosis).

Putative transformants were transferred to new media and/or transferred to potting soil in the greenhouse.

### DNA Analyses

DNA was isolated from in vitro shoots by a CTAB DNA isolation protocol as described by Rogers and Bendich (1988). PCRs were performed with primers NPT1 and NPT2, trfA1 and trfA2, insB1 and insB2 (Sequence nos. 1, 2, 5, 6, 7 and 8; Wolters et al. 1998a), to study the presence of backbone vector DNA in the transformants.

Four μg of DNA of the transformants was digested with HindIII, fragments were separated by gel electrophoresis, and Southern blots were made. Blots were hybridized with the R3a probe, to check the number of T-DNA insertions.

### Border-Free Transformants

### PCR on the Border Sequences

The right border and left border sequences of the T-DNA are not derived from Solanum sp., but obtained from pBIN19 (pTiT37 borders). To analyze whether these border sequences are present or absent in the transformants PCRs were performed. For the analysis of the right border the following primers were used: primer RBcheck2 (5′-CCAATATATCCTGTCAAACA-3′ (SEQ ID NO: 4)) and primer SHcis1 (5′-CATCATCATCCCAAGTACAA-3′ (SEQ ID NO: 5)). With these primers a fragment of 1221 by was expected when DNA of vector pBINmf::R3a was used as template. For the analysis of the left border primer SHcis2 (5′-AAGGGCAAACATAACCATTC-3′ (SEQ ID NO: 6)) was used in combination with primer LBcheck1 (5′-GGATATATTGTGGTGTAAAC-3′ (SEQ ID NO: 7)). With these primers a fragment of 1712 by was expected when DNA of vector pBINmf::R3a was used as template.

### Universal Genomic Walking

To obtain T-DNA flanking sequences from the transformants the Universal GenomeWalker Kit (Clontech) was used. DNA of the transformants was digested with DraI, EcoRV, PvuII, ScaI and StuI. GenomeWalker Adaptors were ligated to the obtained fragments to create enzyme-specific libraries. To obtain RB-flanking DNA the first PCR was performed with adaptor primer AP1 (5′-GTAATACGACTCACTATAGGGC-3′ (SEQ ID NO: 8)) and primer SHGW1 (5′-TAAGTTTAATCAGAAGTTGGGTAGGAA-3′ (SEQ ID NO: 9)). The nested PCR was performed with adaptor primer AP2 (5′-ACTATAGGGCACGCGTGGT-3′ (SEQ ID NO: 10)) and primer SHGW2 (5′-GTTGGGTAGGAAGCCTGCTCTTGGAAA-3′ (SEQ ID NO: 11)). To obtain LB-flanking DNA the first PCR was performed with adaptor primer AP1 and primer SHGW3 (5′-GTATGTATGTGTAGTTAATGGGGTAGT-3′ (SEQ ID NO: 12)). The nested PCR was performed with adaptor primer AP2 and primer SHGW4 (5′-ACGGTTTCTAAATTAACGTAGCCAATA-3′ (SEQ ID NO: 13)).

### Example 2

### Selection of Cisgene R3a Transformants

Construction of pBINAW2b::R3 with Reduced T-DNA Borders

A new backbone vector including the RB and LB was constructed using pBIN19 as starting material. Primers for the RB and LB were designed. Primers URB (5′-GCGGTCCTGATCAATCGTCAC-3′ (SEQ ID NO: 14)) and RBK (5′-GGTACCTGACAGGATATATTGGCGGGTAAA-3′ (SEQ ID NO: 15); with KpnI site) were used to amplify an RB upstream DNA sequence from pBIN19 of 1156 bp. Primers LBKX (5′-GGTACCTCTAGAGTTTACACCACAATATATCC-3′ (SEQ ID NO: 16); with KpnI and XbaI sites) and DLB (5′-GCGGGTTTAACCTACTTCCTTT-3′ (SEQ ID NO: 17)) were used to amplify an LB downstream DNA sequence from pBIN19 of 627 bp. Both PCR products were cloned into pGEM-T, and sequenced. The RB upstream sequence was released from the pGEM-T vector by digestion with SacI and KpnI. The LB downstream sequence was released from the pGEM-T vector by digestion with KpnI and NsiI. Fragments were isolated from agarose gels. The SacI/KpnI RB upstream sequence and the KpnI/NsiI LB downstream sequence were ligated into SacI/NsiI digested pUC28 vector (Bene\check{s} et al., 1993), resulting in plasmid pUC28-LBRB. Through this ligation the RB and LB were ligated to each other, separated only by a KpnI and an XbaI recognition sequence. A 644-bp BglII/Nsi fragment was released from this vector, and ligated to the 6966-bp BclI/NsiI backbone vector sequence from pBIN19, resulting in plasmid pBINAW2. By this ligation the tetR gene flanking the RB was deleted.

To obtain a vector with more than two unique restriction sites between the RB and LB plasmid pUC28-LBRB was digested with KpnI, and subsequently self-ligated, resulting in plasmid pUC28-LBRB1. A 562-bp XbaI fragment from plasmid pUC28-LBRB was ligated into XbaI-digested pUC28-LBRB1, resulting in plasmid pUC28-LBRB2. This plasmid contains the RB upstream sequence and the LB downstream sequence, separated by a multiple cloning site for KpnI, SmaI, BamHI and XbaI. Plasmid pUC28-LBRB2 was digested with KpnI and NsiI. The 526-bp fragment was isolated from an agarose gel and ligated into KpnI/NsiI-digested pBINAW2, resulting in vector pBINAW2a. A double stranded oligo was made by annealing primers MNO1 (5′-CGGCGCGCCCGGGTTAATTAAG-3′ (SEQ ID NO: 18)) and MNO2 (5′-GATCCTTAATTAACCCGGGCGCGCCGGTAC-3′ (SEQ ID NO: 19)). This sequence contains KpnI and BamHI sticky ends and restriction sites for AscI, SmaI and PacI. The oligo was ligated into KpnI/BamHI-digested pBINAW2a, resulting in vector pBINAW2b.

Vector pBINAW2b::R3a was developed by the cloning of a PacI/AscI genomic DNA fragment of SH23-2 into PacI/AscI-digested pBINAW2b. SH23-2 is a subclone of the Bacterial Artificial Chromosome (BAC) SH23 (Huang et al. 2005). This BAC was partially digested with Sau3AI and the 7-10 kb fraction was ligated into the BamHI site of vector pBINPLUS.

The T-DNA in vector pBINAW2b::R3a contains a 9-kb fragment of the SH23-2 pBINPLUS subclone, in which the Coding Sequence (CDS) of gene R3a is situated. The 3849-bp CDS is (after transformation of the plant) regulated by the original promoter and terminator of R3a which are both present on SH23-2 (FIG. 5). The genomic fragment SH23-2 is isolated from the diploid potato clone SH83-92-488 (Huang et al. 2005).

The binary vector pBINAW2b::R3a was transformed into Agrobacterium tumefaciens strain AGL-1 by triparental mating, using helper plasmid pRK2013 (Figurski and Helinski, 1979).

### Transformation of Potato and Selection of Transformants

Internodal cuttings or leaves from in vitro grown plants of potato cultivar ‘Desiree’ were used for transformation by Agrobacterium tumefaciens co-cultivation, according to the protocol described by Visser et al. (1991a).

Potato cultivar ‘Desiree’ was transformed with pBINAW2b::R3a in A. tumefaciens COR308 or AGL-1. No selection was performed. After four weeks the first shoots were harvested and harvesting of shoots continued for about three months. No more than two regenerants per stem explant were harvested and shoots were allowed to grow on MS30 medium in a glass jars (diameter 10 cm) or plastic jars (diameter 15 cm). Each jar contained 8 cuttings. A 2 to 3 cm space was left between the cuttings and the inner wall of the jars.

PCR Selection of Desiree R3a transformants.

After 1 to 2 weeks leaf or stem material of 8 or more independent shoots was harvested and pooled (pool size depended on the frequency of transformants expected). DNA of these pools of shoots was isolated in 96-wells microtiter plates using a CTAB DNA isolation protocol as described by Rogers and Bendich (1988).

PCR analyis was performed on DNA isolated from the pools of regenerants with the primers AL79 (5′-GAGAATGGAAGATTTGGGTGAAG-3′ (SEQ ID NO: 20)) and AL80 (5′-CTAATCTCACCAGTTGGCTGTTC-3′ (SEQ ID NO: 21)), to check for the presence of transformants. Of the PCR positive pools leaf and/or stem material of individual shoots was harvested in 96-wells microtiter plates and genomic DNA was isolated using the CTAB genomic DNA isolation procedure. PCR analyis was performed on DNA isolated from the individual regenerants with the primers AL79 (5′-GAGAATGGAAGATTTGGGTGAAG-3′ (SEQ ID NO: 20)) and AL80 (5′-CTAATCTCACCAGTTGGCTGTTC-3′ (SEQ ID NO: 21)), to check for the presence of transformants.

In Vitro Selection with avr3

In vitro plantlets were grown at 24° C. and 16 h light/8 h dark. Plantlets with 3 to 4 fully developed leaves were used for in vitro inoculation. Depending on the potato genotype, after 2 to 4 weeks plantlet were ready for inoculation.

The 3 largest leaves of each in vitro plantlet were inoculated (1 spot per leaf) by pipetting 10 μl droplets of a zoospore suspension of 2.5×10⁴ spores/ml on the adaxial side. Inoculum preparation and inoculation were performed in sterilized conditions. Leaves touching the inner wall or the lid of the jars or the medium were excluded because we found these leaves to be more susceptible than others of the same plants. The jars with inoculated in vitro plantlets were incubated in the same climate chamber as the trays with inoculated detached leaves.

Non-transformed plantlet showing a susceptible interactions were scored as S (spreading lesion with massive sporulation) or class SQ (spreading lesion with no or little sporulation). Putative transformants showing a resistant interaction were scored as class R (no symptom or localized HR-like necrosis) or class RQ (trailing HR necrosis).

Putative transformants were transferred to new media and/or transferred to potting soil in the greenhouse.

Efficiency of Marker-Free R3a Transformation of Potato Cv. Desiree

Approximately 5,000 stem internode or leaf explants of potato cultivar ‘Desiree’ were inoculated with pBINAW2b::R3a in A. tumefaciens COR308 or in A. tumefaciens Agl-1. The number of shoots harvested, the frequency of PCR-positive shoots obtained, and the frequency of shoots showing a phenotype are shown in Table 2.

With COR 308 none of the harvested shoots were scored PCR-positive, whereas with AGL-1 this percentage was more than 1.5%. Of these PCR-positive transformants more than 60% showed R3a resistance.

Thus, transformation with a highly virulent Agrobacterium strain AGL1 results in a large number of PCR positive shoots of which more than 60% expresses the phenotype.

TABLE 2
Efficient marker-free transformation of the R3a gene into
Desiree potato with the highly virulent AGL1 Agrobacterium strain.

| | No. of | No. of PCR-positive | |
|---|---|---|---|
| A. tum | shoots | plants (% of total | No. of R3a plants (% of |
| strain | harvested | shoots) | PCR-positive clones) |
| COR308 | 2000 | 0 (0%) | nd |
| AGL1 | 3728 | 56 (1.5%) | 38 (68%) |

### DNA Analyses

DNA was isolated from in vitro shoots by a CTAB DNA isolation protocol as described by Rogers and Bendich (1988). PCRs were performed with primers NPT1 and NPT2, trfA1 and trfA2, insB1 and insB2 (Sequence nos. 1, 2, 5, 6, 7 and 8; Wolters et al. 1998a), to study the presence of backbone vector DNA in the transformants.

Four μg of DNA of the transformants was digested with HindIII, fragments were separated by gel electrophoresis, and Southern blots were made. Blots were hybridized with the R3a probe, to check the number of T-DNA insertions.

### Selection of Single-Copy and Vector DNA-Free Transformants

One of the requirements for a intragenic or cisgenic transformant should be that it does not possess vector DNA or multiple inserts of the T-DNA. Integration of DNA beyond the borders into the genome of the host plants is reported to occur in 20 to 75% of the transformed plants. Selected marker-free R3a resistant potato transformants were analysed for presence of backbone vector DNA by PCR using primers to four open reading frames of the pBIN19 vector. Of the 38 tested R3a resistant transformants 20 were negative for all five DNA fragments. These 20 vector DNA-free transformants were further analysed by Southern blot hybridisation using R3a as probe. These analyses showed that most transformants contain 3 or less copies of the T-DNA insertion. Moreover, of the 20 transformants analysed 8 contained a single T-DNA insertion.

This demonstrates that it is feasible to obtain R3a transformants without the use of selection marker genes free of backbone vector DNA, containing only one insert of the T-DNA.

### Border-Free Transformants

### PCR on the Border Sequences

The right border and left border sequences of the T-DNA are not derived from Solanum sp., but obtained from pBIN19 (pTiT37 borders). To analyze whether these border sequences are present or absent in the transformants PCRs were performed. For the analysis of the right border the following primers were used: primer RBcheck1 (5′-CCAATATATCCTGTCAGGTA-3′ (SEQ ID NO: 22)) and primer SHcis1 (5′-CATCATCATCCCAAGTACAA-3′ (SEQ ID NO: 5)). With these primers a fragment of 795 by was expected when DNA of vector pBINAW2b::R3a was used as template. For the analysis of the left border primer SHcis2 (5′-AAGGGCAAACATAACCATTC-3′ (SEQ ID NO: 6)) was used in combination with primer LBcheck1 (5′-GGATATATTGTGGTGTAAAC-3′ (SEQ ID NO: 7)). With these primers a fragment of 1084 by was expected when DNA of vector pBINAW2b::R3a was used as template.

### Universal Genomic Walking

To obtain T-DNA flanking sequences from the transformants the Universal GenomeWalker Kit (Clontech) was used. DNA of the transformants was digested with DraI, EcoRV, PvuII, ScaI and StuI. GenomeWalker Adaptors were ligated to the obtained fragments to create enzyme-specific libraries. To obtain RB-flanking DNA the first PCR was performed with adaptor primer AP1 (5′-GTAATACGACTCACTATAGGGC-3′ (SEQ ID NO: 8)) and primer SHGW1 (5′-TAAGTTTAATCAGAAGTTGGGTAGGAA-3′ (SEQ ID NO: 9)). The nested PCR was performed with adaptor primer AP2 (5′-ACTATAGGGCACGCGTGGT-3′ (SEQ ID NO: 10)) and primer SHGW2 (5′-GTTGGGTAGGAAGCCTGCTCTTGGAAA-3′ (SEQ ID NO: 11)). To obtain LB-flanking DNA the first PCR was performed with adaptor primer AP1 and primer SHGW3 (5′-GTATGTATGTGTAGTTAATGGGGTAGT-3′ (SEQ ID NO: 12)). The nested PCR was performed with adaptor primer AP2 and primer SHGW4 (5′-ACGGTTTCTAAATTAACGTAGCCAATA-3′ (SEQ ID NO: 13)).

### Selection of Cisgenic R3a Transformants

The 8 marker-free, vector DNA free, single copy R3a transformants were analysed for the absence of border sequences using the Genome walking kit as described above. 6 out of 8 transformants did not contain any right border sequences, whereas 5 out of 8 did not contain any left border sequences. Deletions up to 500 by were observed. Of the 8 transformants 4 did not contain any Agrobacterium derived T-DNA border sequences. These transformants are cisgenic according to the definition that they do not contain sequences derived from other species than potato and these transformants have an insertion as in the native genomic context as one could find in the original potato species where this gene is found i.e. Solanum demissum.

### Example 3

### Marker-Free GBSS-IR Construct

### Construction of an all Potato GBSS-IR T-DNA Vector.

The T-DNA sequence is completely derived from potato GBSSI genomic sequences. To clone an extended GBSSI promoter the sequence upstream of the HindIII site of the commonly used promoter (Visser et al., 1991b; van der Leij et al., 1991a; accession number X58453) until the BglII site 0.6 kb upstream (van der Leij et al., 1991a) was, determined. For this a PCR was performed with primers GBSS-0 (5′-TACCGCTACCACTTGACATTC-3′ (SEQ ID NO: 25)) and BINMCS (5′-GCACCCCAGGCTTTACACTTT-3′ (SEQ ID NO: 26)) using DNA of plasmid pWAM101 (van der Leij et al., 1991b) as template. The 738-bp sequence between BglII and HindIII was highly homologous (98% identical) to the sequence published by Dai et al. (1996), accession number X83220.

Primers were designed to amplify the GBSSI promoter and upstream region: primer UPGBX (5′-CTCTAGAAGTTCGAGACACTGGCTACG-3′ (SEQ ID NO: 27); with XbaI site) and primer PGBB (5′-GGATCCTGGAGGAGATGAGTAAAAGTTA-3′ (SEQ ID NO: 28); with BamHI site). These primers were used in a PCR with pWAM200 DNA as template. Vector pWAM200 contains the same 6.5-kb BglII fragment of GBSSI genomic DNA as vector pWAM100 (van der Leij et al., 1991a), but cloned in pMTL24 (Chambers et al., 1988). The 1528-bp PCR product was cloned in pGEM-T and sequenced.

For the GBBSI terminator and downstream sequences primers were designed on the basis of the sequence published by van der Leij et al. (1993) (accession number X66826). Primers TGBB (5′-GGATCCAAACGTATTTACTAGCGAACT-3′ (SEQ ID NO: 29); with BamHI site) and DTGBK (5′-GGTACCAAAGAGACAGGTGCCGTTAT-3′ (SEQ ID NO: 30); with KpnI site) amplified a 658-bp PCR product containing the GBSSI terminator plus downstream sequences from plasmid pWAM200. This PCR product was cloned into pGEM-T and sequenced.

The extended GBSSI promoter was released from the pGEM-T vector by digestion with XbaI and BamHI. The extended GBSSI terminator was released from the pGEM-T vector by digestion with BamHI and KpnI. Both fragments were ligated into XbaI/KpnI-digested pUC28 (Bene\check{s} et al., 1993), resulting in plasmid pUC28-PTGB. Subsequently, plasmid pUC28-PTGB was digested with XbaI and KpnI, and the released fragment was ligated into XbaI/KpnI-digested pBINAW2a vector (see Example 1). In the resulting vector pBINAW3 the extended GBSSI promoter is flanked by the LB and the extended GBSSI terminator is flanked by the RB.

A 1390-bp BamHI fragment containing an inverted repeat of the middle part of the GBSSI cDNA, with a spacer sequence consisting of potato GBSSI cDNA was isolated from vector pIRMAS (Heilersig et al., 2006). This fragment was cloned into BamHI-digested pBINAW3. The resulting binary vector pBINAW4 contained an antisense-sense inverted repeat of the GBSSI gene between an extended GBSSI promoter and an extended GBSSI terminator flanked immediately by the LB and RB sequences of pBIN19.

The binary vector pBINAW4 was transformed into Agrobacterium tumefaciens strain AGL-1 by triparental mating, using helper plasmid pRK2013 (Figurski and Helinski, 1979).

### Transformation of Potato and Selection of Transformants

Internodal cuttings of leaves from in vitro grown plants of potato cultivars ‘Aventra’ or ‘Aveka’ were used for transformation by Agrobacterium tumefaciens co-cultivation, according to the protocol described by Visser (1991a).

No selection was performed. After four weeks the first shoots were harvested and harvesting of shoots continued for about three months. No more than two regenerants per stem explant were harvested and shoots were allowed to grow on MS30 medium in a glass jars (diameter 10 cm) or plastic jars (diameter 15 cm). Each jar contained 8 cuttings. A 2 to 3 cm space was left between the cuttings and the inner wall of the jars.

### PCR Selection of Aveka GBSS-IR Transformants

After 1 to 2 weeks leaf or stem material of 8 or more independent shoots was harvested and pooled (pool size depended on the frequency of transformants expected). DNA of these pools of shoots was isolated in 96-wells microtiter plates using a CTAB DNA isolation protocol as described by Rogers and Bendich (1988).

PCR analyis was performed on DNA isolated from the pools of regenerants with the primers AMYML F (5′-AGA TAA GCT TTC TCA TTC CTT GC-3′ (SEQ ID NO: 31)) and AMYML R (5′-TCC TCC AGG ATC CTT CTG G-3′ (SEQ ID NO: 32)), to check for the presence of transformants. Of the PCR positive pools leaf and/or stem material of individual shoots was harvested in 96-wells microtiter plates and genomic DNA was isolated using the CTAB genomic DNA isolation procedure. PCR analyis was performed on DNA isolated from the individual regenerants with the primers AMYML F (5′-AGA TAA GCT TTC TCA TTC CTT GC-3′ (SEQ ID NO: 31)) and AMYML R (5′-TCC TCC AGG ATC CTT CTG G-3′ (SEQ ID NO: 32)), to check for the presence of transformants.

### In Vitro Tuberization

To PCR-positive shoots 20 ml of a liquid medium was added, consisting of KI medium (‘knolinducerend’ (=tuber inducing) medium, Duchefa) with 325 g/L sucrose and 1.75 g/L CCC (chlorocholine chloride, or cyclocel.). The pots were placed in a dark growth chamber at a temperature of 18° C. After 2 to 4 weeks microtubers had developed on most shoots.

### Starch Staining

Microtubers were cut and stained with an iodine solution to assess the presence of amylose in the starch granules. Staining of the starch granules was inspected with a microscope.

Efficiency of Marker-Free GBSS-IR Transformation of Potato Cv. Aveka

Approximately 2000 stem internode or leaf explants of potato cultivar ‘Aveka’ were inoculated with pBINAW4 in A. tumefaciens AGL-1. A total of 3787 shoots have been harvested, of which 28 PCR-positive shoots were obtained (0.74%). Of these 28 shoots 72% contained the amylose-free phenotype.

### DNA Analyses

DNA was isolated from in vitro shoots by a CTAB DNA isolation protocol as described by Rogers and Bendich (1988). PCRs were performed with primers NPT1 and NPT2, trfA1 and trfA2, insB1 and insB2 (Sequence nos. 1, 2, 5, 6, 7 and 8; Wolters et al. 1998a), to study the presence of backbone vector DNA in the transformants.

Four μg of DNA of the transformants was digested with BamHI, HindIII and EcoRI, fragments were separated by gel electrophoresis, and Southern blots were made. Blots were hybridized with a GBSS terminator probe, to check the number of T-DNA insertions.

### Selection of Single Copy, Vector DNA-Free Transformants

One of the requirements for an intragenic transformant should be that it does not possess vector DNA or multiple inserts of the T-DNA. The 18 selected marker-free amylose-free “Aveka” transformants were analysed for presence of backbone vector DNA by PCR using primers to four open reading frames of the pBIN19 vector. Of the 18 tested R3a resistant transformants 8 were negative for all five DNA fragments. These 8 vector DNA-free transformants were further analysed by Southern blot hybridisation using the GBSS terminator sequence as probe. These analyses showed that all transformants contained 1 or 2 copies of the T-DNA insertion. Moreover, of the 8 transformants analysed 5 contained a single T-DNA insertion.

This demonstrates that it is feasible to obtain GBSS-IR transformants without the use of selection marker genes free of backbone vector DNA, containing only one insert of the T-DNA.

### Border-Free Transformants

### PCR on the Border Sequences

The right border and left border sequences of the T-DNA are not derived from Solanum sp., but obtained from pBIN19 (pTiT37 borders). To analyze whether these border sequences are present or absent in the transformants PCRs were performed. For the analysis of the left and right border deletions the following primersets were used:

### For Right Border Analysis

| | |
|---|---|
| Amy ML LB: | |
| 5′-CAGGATATATTGTGGTGTAAACTC-3′ | (SEQ ID NO: 37) |
| in combination with GBSS23: | |
| 5′-TCAATGTTTGTTACATTTCTTCC-3′ | (SEQ ID NO: 43) |
| Amy ML prom 1: | |
| 5′-TATCTTTGCTCAGGACCCTG-3′ | (SEQ ID NO: 38) |

in combination with Amy ML as-prom 1:

| | |
|---|---|
| 5′-GCAGAAGGATGCTGGAGG-3′ | (SEQ ID NO: 39) |
| Amy ML prom2: | |
| 5′-AACTCGAAGTCAGCCTGCG-3′ | (SEQ ID NO: 40) |

in combination with Amy ML as-prom 1:

| | |
|---|---|
| 5′-GCAGAAGGATCCTGGAGG-3′ | (SEQ ID NO: 39) |
| GBSS9: | |
| 5′-CAAATGCAACAGTATCTTGTACC-3′ | (SEQ ID NO: 42) |
| in combination with GBSS0: | |
| 5′-ACCGCTACCACTTGACATTCC-3′ | (SEQ ID NO: 41) |

### For Left Border Analysis

| | |
|---|---|
| Amy ML RB: | |
| 5′-TCAGGTACCAAAGAGACAGG-3′ | (SEQ ID NO: 33) |

in combination with Amy ML sense term:

| | |
|---|---|
| 5′-GGAGCAGAAGGATCCAAACG-3′ | (SEQ ID NO: 34) |
| Amy ML term 1: | |
| 5′-TGCCGTTATGTAAAGGAG-3′ | (SEQ ID NO: 35) |

in combination with Amy ML sense term:

| | |
|---|---|
| 5′-GGAGCAGAAGGATCCAAACG-3′ | (SEQ ID NO: 34) |
| Amy ML term2: | |
| 5′-AGCTTCTTTCATATGACCAACC-3′ | (SEQ ID NO: 36) |

in combination with Amy ML sense term:

| | |
|---|---|
| 5′-GGAGCAGAAGGATCCAAACG-3′ | (SEQ ID NO: 34) |

### Universal Genomic Walking

To obtain T-DNA flanking sequences from the transformants the Universal GenomeWalker Kit (Clontech) was used. DNA of the transformants was digested with DraI, EcoRV, PvuII, ScaI and StuI. GenomeWalker Adaptors were ligated to the obtained fragments to create enzyme-specific libraries. To obtain RB-flanking DNA the first PCR was performed with adaptor primer AP1 (5′-GTAATACGACTCACTATAGGGC-3′ (SEQ ID NO: 8)) and primer SHGW1 (5′-TAAGTTTAATCAGAAGTTGGGTAGGAA-3′ (SEQ ID NO: 9)). The nested PCR was performed with adaptor primer AP2 (5′-ACTATAGGGCACGCGTGGT-3′ (SEQ ID NO: 10)) and primer SHGW2 (5′-GTTGGGTAGGAAGCCTGCTCTTGGAAA-3′ (SEQ ID NO: 11)). To obtain LB-flanking DNA the first PCR was performed with adaptor primer AP1 and primer GBSSIGW1 (5′-TTTACTCATCTCCTCCAGGATCCTTCT-3′ (SEQ ID NO: 45)). The nested PCR was performed with adaptor primer AP2 and primer GBSSIGW2 (5′-ATCTCCTCCAGGATCCTTCTGCTCCTC-3′ (SEQ ID NO: 46)).

### Selection of Intragenic GBSS-IR Transformants

The 5 marker-free, vector DNA free, single copy GBSS-IR transformants were analysed for the absence of border sequence using PCR analysis as described above. 2 out of 5 transformants did not contain any right border sequences, whereas these transformant showed large deletions of left border sequence. Of the 5 transformants analysed 2 did not contain any Agrobacterium derived T-DNA border sequences. These transformants are intragenic according to the definition that they do not contain sequences derived from other species than potato (see FIG. 7).

### Example 4

### Marker-Free R3a-GBSS-IR Construct

Construction of an all Potato R3a-GBSS-IR T-DNA Vector.

The T-DNA sequence is completely derived from potato GBSSI genomic sequences. To clone an extended GBSSI promoter the sequence upstream of the HindIII site of the commonly used promoter (Visser et al., 1991b; van der Leij et al., 1991a; accession number X58453) until the BglII site 0.6 kb upstream (van der Leij et al., 1991a) was determined. For this a PCR was performed with primers GBSS-0 (5′-TACCGCTACCACTTGACATTC-3′ (SEQ ID NO: 25)) and BINMCS (5′-GCACCCCAGGCTTTACACTTT-3′ (SEQ ID NO: 26)) using DNA of plasmid pWAM101 (van der Leij et al., 1991b) as template. The 738-bp sequence between BglII and HindIII was highly homologous (98% identical) to the sequence published by Dai et al. (1996), accession number X83220.

Primers were designed to amplify the GBSSI promoter and upstream region: primer UPGBX (5′-CTCTAGAAGTTCGAGACACTGGCTACG-3′ (SEQ ID NO: 27); with XbaI site) and primer PGBB (5′-GGATCCTGGAGGAGATGAGTAAAAGTTA-3′ (SEQ ID NO: 28); with BamHI site). These primers were used in a PCR with pWAM200 DNA as template. Vector pWAM200 contains the same 6.5-kb BglII fragment of GBSSI genomic DNA as vector pWAM100 (van der Leij et al., 1991a), but cloned in pMTL24 (Chambers et al., 1988). The 1528-bp PCR product was cloned in pGEM-T and sequenced.

For the GBBSI terminator and downstream sequences primers were designed on the basis of the sequence published by van der Leij et al. (1993) (accession number X66826). Primers TGBB (5′-GGATCCAAACGTATTTACTAGCGAACT-3′ (SEQ ID NO: 29); with BamHI site) and DTGBK (5′-GGTACCAAAGAGACAGGTGCCGTTAT-3′ (SEQ ID NO: 30); with KpnI site) amplified a 658-bp PCR product containing the GBSSI terminator plus downstream sequences from plasmid pWAM200. This PCR product was cloned into pGEM-T and sequenced.

The extended GBSSI promoter was released from the pGEM-T vector by digestion with XbaI and BamHI. The extended GBSSI terminator was released from the pGEM-T vector by digestion with BamHI and KpnI. Both fragments were ligated into XbaI/KpnI-digested pUC28 (Bene\check{s} et al., 1993), resulting in plasmid pUC28-PTGB. Subsequently, plasmid pUC28-PTGB was digested with XbaI and KpnI, and the released fragment was ligated into XbaI/KpnI-digested pBINAW2a vector (see Example 1). In the resulting vector pBINAW3 the extended GBSSI promoter is flanked by the LB and the extended GBSSI terminator is flanked by the RB.

A 1390-bp BamHI fragment containing an inverted repeat of the middle part of the GBSSI cDNA, with a spacer sequence consisting of potato GBSSI cDNA was isolated from vector pIRMAS (Heilersig et al., 2006). This fragment was cloned into BamHI-digested pBINAW3. The resulting binary vector pBINAW4 contained an antisense-sense inverted repeat of the GBSSI gene between an extended GBSSI promoter and an extended GBSSI terminator flanked immediately by the LB and RB sequences of pBIN19.

Vector pBINAW4a was derived from vector pBINAW4. A double-stranded oligo with two KpnI sticky ends was ligated into the KpnI site of pBINAW4. This double-stranded oligo harbours restriction sites for AscI, SmaI and PvuI. Two orientations of the oligo are possible: one with the AscI site closest to the RB, and one with the PvuI site closest to the RB. The first orientation was selected.

pBINAW4a::R3a was developed by the cloning of a PacI/AscI genomic DNA fragment of SH23-2 into PvuI/AscI-digested pBINAW4a. SH23-2 is a subclone of the Bacterial Artificial Chromosome (BAC) SH23 (Huang et al. 2005). This BAC was partially digested with Sau3AI and the 7-10 kb fraction was ligated into the BamHI site of vector pBINPLUS. Using the restriction enzymes Pad and AscI, a 9-kb fragment (truncated SH23-2) was cut out of pBINPLUS::R3a and the fragment was ligated into the PvuI and AscI sites of pBINAW4a.

The T-DNA contains a fragment of SH23-2, in which the Coding Sequence (CDS) of the R3a gene is present. This CDS is (after transformation of the plant) regulated by the original promoter and terminator of R3a which are also present on SH23-2. The genomic fragment SH23-2 is isolated from the diploid potato clone SH83-92-488 (Huang et al., 2005).

The binary vector pBINAW4a::R3a (FIG. 6) was transformed into Agrobacterium tumefaciens strain AGL-1 by triparental mating, using helper plasmid pRK2013 (Figurski and Helinski, 1979).

### Transformation of Potato and Selection of Transformants

Internodal cuttings from in vitro grown plants of potato cultivars ‘Desiree’, ‘Aventra’ or ‘Aveka’ were used for transformation by Agrobacterium tumefaciens co-cultivation, according to the protocol described by Visser (1991a).

Potato cultivar ‘Desiree’ was transformed with pBINAW4a::R3a in A. tumefaciens AGL-1 according to the same protocol. No selection was performed. After four weeks the first shoots were harvested and harvesting of shoots continued for about three months. No more than two regenerants per stem explant were harvested and shoots were allowed to grow on MS30 medium in a glass jars (diameter 10 cm) or plastic jars (diameter 15 cm). Each jar contained 5 cuttings. A 2 to 3 cm space was left between the cuttings and the inner wall of the jars.

### In Vitro Assay for Disease Testing

In vitro plantlets were grown at 24° C. and 16 h light/8 h dark. Plantlets with 3 to 4 fully developed leaves were used for in vitro inoculation. Depending on the potato genotype, after 2 to 4 weeks plantlet were ready for inoculation.

The 3 largest leaves of each in vitro plantlet were inoculated (1 spot per leaf) by pipetting 10 μl droplets of a zoospore suspension of 2.5×10⁴ spores/ml on the adaxial side. Inoculum preparation and inoculation were performed in sterilized conditions. Leaves touching the inner wall or the lid of the jars or the medium were excluded because we found these leaves to be more susceptible than others of the same plants. The jars with inoculated in vitro plantlets were incubated in the same climate chamber as the trays with inoculated detached leaves.

Non-transformed plantlet showing a susceptible interactions were scored as S (spreading lesion with massive sporulation) or class SQ (spreading lesion with no or little sporulation). Putative transformants showing resistant interactions were scored as class R (no symptom or localized HR-like necrosis) or class RQ (trailing HR necrosis).

Putative transformants were transferred to new media and/or transferred to potting soil in the greenhouse.

### In Vitro Tuberization

To PCR-positive shoots 20 ml of a liquid medium was added, consisting of KI medium (‘knolinducerend’ (=tuber inducing) medium, Duchefa) with 325 g/L sucrose and 1.75 g/L CCC (chlorocholine chloride, or cyclocel.). The pots were placed in a dark growth chamber at a temperature of 18° C. After 2 to 4 weeks microtubers had developed on most shoots.

### Starch Staining

Microtubers were cut and stained with an iodine solution to assess the presence of amylose in the starch granules. Staining of the starch granules was inspected with a microscope.

### DNA Analyses

DNA was isolated from in vitro shoots by a CTAB DNA isolation protocol as described by Rogers and Bendich (1988). PCRs were performed with primers NPT1 and NPT2, trfA1 and trfA2, insB1 and insB2 (Sequence nos. 1, 2, 5, 6, 7 and 8; Wolters et al. 1998a), to study the presence of backbone vector DNA in the transformants.

Four μg of DNA of the transformants was digested with HindIII, fragments were separated by gel electrophoresis, and Southern blots were made. Blots were hybridized with an R3a probe, to check the number of T-DNA insertions.

### Border-Free Transformants

### PCR on the Border Sequences

The right border and left border sequences of the T-DNA are not derived from Solanum sp., but obtained from pBIN19 (pTiT37 borders). To analyze whether these border sequences are present or absent in the transformants PCRs were performed. For the analysis of the right border the following primers were used: primer RBcheck1 (5′-CCAATATATCCTGTCAGGTA-3 (SEQ ID NO: 22)’) and primer SHcis1 (5′-CATCATCATCCCAAGTACAA-3′ (SEQ ID NO: 5)). With these primers a fragment of 795 by was expected when DNA of vector pBINAW4a::R3a was used as template. For the analysis of the left border primer GBSSIcis1 (5′-CTCTGTCAACAGCCAAATAG-3′ (SEQ ID NO: 44)) was used in combination with primer LBcheck1 (5′-GGATATATTGTGGTGTAAAC-3′ (SEQ ID NO: 7)). With these primers a fragment of 836 by was expected when DNA of vector pBINAW4a::R3a was used as template.

### Universal Genomic Walking

To obtain T-DNA flanking sequences from the transformants the Universal GenomeWalker Kit (Clontech) was used. DNA of the transformants was digested with DraI, EcoRV, PvuII, ScaI and StuI. GenomeWalker Adaptors were ligated to the obtained fragments to create enzyme-specific libraries. To obtain RB-flanking DNA the first PCR was performed with adaptor primer AP1 (5′-GTAATACGACTCACTATAGGGC-3′ (SEQ ID NO: 8)) and primer SHGW1 (5′-TAAGTTTAATCAGAAGTTGGGTAGGAA-3′ (SEQ ID NO: 9)). The nested PCR was performed with adaptor primer AP2 (5′-ACTATAGGGCACGCGTGGT-3′ (SEQ ID NO: 10)) and primer SHGW2 (5′-GTTGGGTAGGAAGCCTGCTCTTGGAAA-3′ (SEQ ID NO: 11)). To obtain LB-flanking DNA the first PCR was performed with adaptor primer AP1 and primer GBSSIGW1 (5′-TTTACTCATCTCCTCCAGGATCCTTCT-3′ (SEQ ID NO: 45)). The nested PCR was performed with adaptor primer AP2 and primer GBSSIGW2 (5′-ATCTCCTCCAGGATCCTTCTGCTCCTC-3′ (SEQ ID NO: 46)).

### Example 5

### Marker-Free Cisgene R3a-blb1 Construct

Vector pBINAW2b::blb1-R3a is a binary plasmid without a selection gene in the T-DNA, and is derived from vector pBINAW2b::R3a. Vector pBINAW2b::R3a was developed by cloning the SH23-2 fragment with the restriction enzymes Pad and AscI from a subclone, and ligating it into the Pad and AscI sites of pBINAW2b (see Example 1). SH23-2 is a pBINPLUS subclone developed by subcloning the Bacterial Artificial Chromosome (BAC) SH23 (Huang et al. 2005). This BAC was partially digested with Sau3AI and the 7-10 kb fraction was ligated into the BamHI site of pBINPLUS. After the development of pBINAW2b::R3a, a RGC2 fragment was placed between the AscI site and the right border using the restriction enzymes AscI and SbfI. RGC2 is a pBINPLUS subclone and is, just like SH23-2, developed by cloning the 7-10 kb fraction of Sau3AI partially digested BAC (SPB4) (van der Vossen et al., 2003) in the BamHI site of pBINPLUS.

A 6.5-kb fragment of RGC2 was amplified using the Polymerase Chain Reaction (PCR). The used primers were extended with an AscI and SbfI site. The amplified fragment could therefore be digested with these enzymes and this fragment was then ligated into the AscI and SbfI sites of pBINAW2b::R3a.

The total pBINAW2b::blb1-R3a construct has a size of 23,147 bp. The T-DNA is flanked by borders; Left Border (LB) and Right Border (R). These borders originate from Agrobacterium and serve as a signal for Agrobacterium to discriminate the border between vector and T-DNA. The T-DNA is integrated by Agrobacterium into the potato genome.

The T-DNA contains a fragment of the SH23-2 and RGC2 pBINPLUS sub-clones, in which the Coding Sequence (CDS) of R3a is situated on SH23-2 and the CDS of Rpi-blb1 is situated on RGC2. These CDS are (after transformation of the plant) regulated by the original promoter and terminator of R3a and Rpi-blb1 which are also present on both SH23-2 and RGC2. The genomic fragment SH23-2 is isolated from the diploid potato clone SH83-92-488 (Huang et al. 2005) and the genomic fragment RGC2 is isolated from Solanum bulbocastanum.

There is no selection gene (like NPTII) present in the T-DNA for the selection of transgenic plants. Only the genomic SH23-2 fragment, the genomic RGC2 fragment and the border sequences will be integrated into the plant genome.

The binary vector pBINAW2b::blb1-R3a was transformed into Agrobacterium tumefaciens strain AGL-1 by triparental mating, using helper plasmid pRK2013 (Figurski and Helinski, 1979).

### Transformation of Potato and Selection of Transformants

Internodal cuttings from in vitro grown plants of potato cultivar ‘Desiree’ were used for transformation by Agrobacterium tumefaciens co-cultivation, according to the protocol described by Visser et al. (1991a).

Potato cultivar ‘Desiree’ was transformed with pBINAW2b::blb1-R3a in A. tumefaciens strain AGL-1. No selection was performed. After four weeks the first shoots were harvested and harvesting of shoots continued for about three months. No more than two regenerants per stem explant were harvested and shoots were allowed to grow on MS30 medium in a glass jars (diameter 10 cm) or plastic jars (diameter 15 cm). Each jar contained 5 cuttings. A 2 to 3 cm space was left between the cuttings and the inner wall of the jars.

In Vitro Selection with Avr3

In vitro plantlets were grown at 24° C. and 16 h light/8 h dark. Plantlets with 3 to 4 fully developed leaves were used for in vitro inoculation. Depending on the potato genotype, after 2 to 4 weeks plantlet were ready for inoculation.

The 3 largest leaves of each in vitro plantlet were inoculated (1 spot per leaf) by pipetting 10 μl droplets of a zoospore suspension of 2.5×10⁴ spores/ml on the adaxial side. Inoculum preparation and inoculation were performed in sterilized conditions. Leaves touching the inner wall or the lid of the jars or the medium were excluded because we found these leaves to be more susceptible than others of the same plants. The jars with inoculated in vitro plantlets were incubated in the same climate chamber as the trays with inoculated detached leaves.

Non-transformed plantlet showing a susceptible interaction were scored as S (spreading lesion with massive sporulation) or class SQ (spreading lesion with no or little sporulation). Putative transformants showing a resistant interaction were scored as class R (no symptom or localized HR-like necrosis) or class RQ (trailing HR necrosis).

Putative transformants were transferred to new media and/or transferred to potting soil in the greenhouse.

Putative transformants were re-analysed for the expression of the blb1 gene. Therefore, either in vitro plants or leaves of greenhouse plants were inoculated with IpO1.

For the detached leaf assay, the 3rd to 5th fully developed leaves (counted from the top) were cut from greenhouse-grown plants and placed in water saturated florists foam (Oasis®, Grünstadt, Germany) in a tray. Depending on the shape and size of the Solanum leaves, one or more leaves were used for inoculation. In general, 10 spots were inoculated per genotype by pipetting 10 μl droplets of a zoospore suspension of 5×10⁴ spores/ml on the abaxial side. The trays were then covered with transparent lids (covered trays), transferred into a climate chamber, and incubated at a 16 h/8 h day/night photoperiod at 16° C.

Resistance level was determined by determining the lesion size (LS), macroscopic scoring, or a combination of both. LS was measured usually at day 6 after spot inoculation using an electronic calliper connected to a computer. The mean LS was calculated from 10 replicates. Based on the average lesion size, a relative score from 0-9 was assigned, relative to Bintje (Table 2). Also the lesion phenotype was examined. In addition macroscopic scoring was carried out as follows.

Compatible interactions were classified as S (susceptible, spreading lesion with massive sporulation) or SQ (spreading lesion with no or little sporulation). Incompatible interactions were scored as R (Resistant, no symptom or localized HR-like necrosis) or RQ (trailing HR necrosis). Also intermediate phenotypes (Q) between compatible and incompatible ones were observed, such as sporulation on 2 to 3 leaflets per compound leaf and localized HR on the other leaflets.

### DNA Analyses

DNA was isolated from in vitro shoots by a CTAB DNA isolation protocol as described by Rogers and Bendich (1988). PCRs were performed with primers NPT1 and NPT2, trfA1 and trfA2, insB1 and insB2 (Sequence nos. 1, 2, 5, 6, 7 and 8; Wolters et al. 1998a), to study the presence of backbone vector DNA in the transformants.

Four μg of DNA of the transformants was digested with HindIII, fragments were separated by gel electrophoresis, and Southern blots were made. Blots were hybridized with the R3a probe, to check the number of T-DNA insertions.

### Border-Free Transformants

### PCR on the Border Sequences

The right border and left border sequences of the T-DNA are not derived from Solanum sp., but obtained from pBIN19 (pTiT37 borders). To analyze whether these border sequences are present or absent in the transformants PCRs were performed. For the analysis of the right border the following primers were used: primer RBcheck1 (5′-CCAATATATCCTGTCAGGTA-3′ (SEQ ID NO: 22)) and primer RGCcis1 (5′-CGCTTTCAGAATCTATTACT-3′ (SEQ ID NO: 47)). With these primers a fragment of 693 by was expected when DNA of vector pBINAW2b::blb1-R3a was used as template. For the analysis of the left border primer SHcis2 (5′-AAGGGCAAACATAACCATTC-3′ (SEQ ID NO: 6)) was used in combination with primer LBcheck1 (5′-GGATATATTGTGGTGTAAAC-3′ (SEQ ID NO: 7)). With these primers a fragment of 1084 by was expected when DNA of vector pBINAW2b::blb1-R3a was used as template.

### Universal Genomic Walking

To obtain T-DNA flanking sequences from the transformants the Universal GenomeWalker Kit (Clontech) was used. DNA of the transformants was digested with DraI, EcoRV, PvuII, ScaI and StuI. GenomeWalker Adaptors were ligated to the obtained fragments to create enzyme-specific libraries. To obtain RB-flanking DNA the first PCR was performed with adaptor primer AP1 (5′-GTAATACGACTCACTATAGGGC-3′ (SEQ ID NO: 8)) and primer RGCGW1 (5′-ATTTCATGCGCATATTCCCGATCAAAC-3′ (SEQ ID NO: 48)). The nested PCR was performed with adaptor primer AP2 (5′-ACTATAGGGCACGCGTGGT-3′ (SEQ ID NO: 10)) and primer RGCGW2 (5′-TCCCGATCAAACTTAAATTACTAGACT-3′ (SEQ ID NO: 49)). To obtain LB-flanking DNA the first PCR was performed with adaptor primer AP1 and primer SHGW3 (5′-GTATGTATGTGTAGTTAATGGGGTAGT-3′ (SEQ ID NO: 12)). The nested PCR was performed with adaptor primer AP2 and primer SHGW4 (5′-ACGGTTTCTAAATTAACGTAGCCAATA-3′ (SEQ ID NO: 13)).

### Example 6

### Marker-Free R3a::Cooking Type

Marker-free transformation-selection for amylose-free-selection of vector DNA, border-free transformants

### Marker-Free R3a::Cooking Type Gene StTLRP

Marker-free transformation-selection for R3a-selection of vector DNA-free, border-free transformants

The coding sequence of cooking type gene StLTRP (Kloosterman 2006) was amplified with PCR primers LTF1 (5′-GGATCCATGGGTTCCAAGGCAATTATGTT-3′ (SEQ ID NO: 50)) and LTR1 (5′-GGATCCGAATGGCTTTATTCATACTTGTT-3′ (SEQ ID NO: 51)). The 360-bp PCR fragment was cloned into pGEM-T vector, and subsequently digested with BamHI. The BamHI insert was isolated from an agarose gel, and ligated to the large BamHI fragment of vector pBINAW4 (see Example 2), thereby replacing the GBSSI cDNA inverted repeat with the StTLRP cDNA sequence. A clone with the StTLRP cDNA in sense orientation between the GBSSI promoter and GBSSI terminator was selected and named pBINAW7.

A double-stranded oligo with two KpnI sticky ends was ligated into the KpnI site of pBINAW7. This double-stranded oligo harbours restriction sites for AscI, SmaI and PvuI. Two orientations of the oligo are possible: one with the AscI site closest to the RB, and one with the PvuI site closest to the RB. The first orientation was selected. The resulting vector was named pBINAW7a. pBINAW7a::R3a was developed by the cloning of a PacI/AscI genomic DNA fragment of SH23-2 into PvuI/AscI-digested pBINAW7a. SH23-2 is a subclone of the Bacterial Artificial Chromosome (BAC) SH23 (Huang et al. 2005). This BAC was partially digested with Sau3AI and the 7-10 kb fraction was ligated into the BamHI site of vector pBINPLUS. Using the restriction enzymes PacI and AscI, a 9-kb fragment (truncated SH23-2) was cut out of pBINPLUS::R3a and the fragment was ligated into the PvuI and AscI sites of pBINAW7a.

The T-DNA contains a fragment of SH23-2, in which the Coding Sequence (CDS) of the R3a gene is present. This CDS is (after transformation of the plant) regulated by the original promoter and terminator of R3a that are also present on SH23-2. The genomic fragment SH23-2 is isolated from the diploid potato clone SH83-92-488 (Huang et al., 2005).

The binary vector pBINAW7a::R3a was transformed into Agrobacterium tumefaciens strain AGL-1by triparental mating, using helper plasmid pRK2013 (Figurski and Helinski, 1979).

### Transformation of Potato and Selection of Transformants

Internodal cuttings from in vitro grown plants of potato cultivars ‘Desiree’, ‘Aventra’ or ‘Aveka’ were used for transformation by Agrobacterium tumefaciens co-cultivation, according to the protocol described by Visser (1991a).

Potato cultivar ‘Desiree’ was transformed with pBINAW4a::R3a in A. tumefaciens AGL-1 according to the same protocol. No selection was performed. After four weeks the first shoots were harvested and harvesting of shoots continued for about three months. No more than two regenerants per stem explant were harvested and shoots were allowed to grow on MS30 medium in a glass jars (diameter 10 cm) or plastic jars (diameter 15 cm). Each jar contained 5 cuttings. A 2 to 3 cm space was left between the cuttings and the inner wall of the jars.

### In Vitro Assay for Disease Testing

In vitro plantlets were grown at 24° C. and 16 h light/8 h dark. Plantlets with 3 to 4 fully developed leaves were used for in vitro inoculation. Depending on the potato genotype, after 2 to 4 weeks plantlet were ready for inoculation.

The 3 largest leaves of each in vitro plantlet were inoculated (1 spot per leaf) by pipetting 10 μl droplets of a zoospore suspension of 2.5×10⁴ spores/ml on the adaxial side. Inoculum preparation and inoculation were performed in sterilized conditions. Leaves touching the inner wall or the lid of the jars or the medium were excluded because we found these leaves to be more susceptible than others of the same plants. The jars with inoculated in vitro plantlets were incubated in the same climate chamber as the trays with inoculated detached leaves.

Non-transformed plantlet showing a susceptible interactions were scored as S (spreading lesion with massive sporulation) or class SQ (spreading lesion with no or little sporulation). Putative transformants showing a resistant interactions were scored as class R (no symptom or localized HR-like necrosis) or class RQ (trailing HR necrosis).

Putative transformants were transferred to new media and/or transferred to potting soil in the greenhouse.

### Analysis of the Cooking-Type

Three tubers of each sample were peeled and steam cooked for 20 minutes after texture was visually scored and categorized on a nominal scale ranging from firm/non-mealy (1) to extremely mealy tubers (6).

### DNA Analyses

DNA was isolated from in vitro shoots by a CTAB DNA isolation protocol as described by Rogers and Bendich (1988). PCRs were performed with primers NPT1 and NPT2, trfA1 and trfA2, insB1 and insB2 (Sequence nos. 1, 2, 5, 6, 7 and 8; Wolters et al. 1998a), to study the presence of backbone vector DNA in the transformants.

Four μg of DNA of the transformants was digested with HindIII, fragments were separated by gel electrophoresis, and Southern blots were made. Blots were hybridized with an R3a probe, to check the number of T-DNA insertions.

### Border-Free Transformants

### PCR on the Border Sequences

The right border and left border sequences of the T-DNA are not derived from Solanum sp., but obtained from pBIN19 (pTiT37 borders). To analyze whether these border sequences are present or absent in the transformants PCRs were performed. For the analysis of the right border the following primers were used: primer RBcheck1 (5′-CCAATATATCCTGTCAGGTA-3′ (SEQ ID NO: 22)) and primer SHcis1 (5′-CATCATCATCCCAAGTACAA-3′ (SEQ ID NO: 5)). With these primers a fragment of 795 bp was expected when DNA of vector pBINAW4a::R3a was used as template. For the analysis of the left border primer GBSSIcis1 (5′-CTCTGTCAACAGCCAAATAG-3′ (SEQ ID NO: 44)) was used in combination with primer LBcheck1 (5′-GGATATATTGTGGTGTAAAC-3′ (SEQ ID NO: 7)). With these primers a fragment of 836 by was expected when DNA of vector pBINAW4a::R3a was used as template.

### Universal Genomic Walking

To obtain T-DNA flanking sequences from the transformants the Universal GenomeWalker Kit (Clontech) was used. DNA of the transformants was digested with DraI, EcoRV, PvuII, ScaI and StuI. GenomeWalker Adaptors were ligated to the obtained fragments to create enzyme-specific libraries. To obtain RB-flanking DNA the first PCR was performed with adaptor primer AP1 (5′-GTAATACGACTCACTATAGGGC-3′ (SEQ ID NO: 8)) and primer SHGW1 (5′-TAAGTTTAATCAGAAGTTGGGTAGGAA-3′ (SEQ ID NO: 9)). The nested PCR was performed with adaptor primer AP2 (5′-ACTATAGGGCACGCGTGGT-3′ (SEQ ID NO: 10)) and primer SHGW2 (5% GTTGGGTAGGAAGCCTGCTCTTGGAAA-3′ (SEQ ID NO: 11)). To obtain LB-flanking DNA the first PCR was performed with adaptor primer AP1 and primer GBSSIGW3 (5′-ATTGCCTTGGAACCCATGGATCCTTCT-3′ (SEQ ID NO: 52)). The nested PCR was performed with adaptor primer AP2 and primer GBSSIGW4 (5′-TGGAACCCATGGATCCTTCTGCTCCTC-3′ (SEQ ID NO: 53)).

### Example 7

### Marker-Free Cisgene R3a GBSS-IR Contruct with Attenuation Region

Construction of an all Potato T-DNA Vector with Sequences Outside the LB to Prevent Integration of Backbone Vector DNA

Integration of backbone vector (non-T-DNA) sequences in the plant genome frequently occurs during Agrobacterium tumefaciens transformation (Kononov et al. 1997; Ramanathan and Veluthambi, 1995; Van der Graaff et al., 1996; Wenck et al., 1997; Wolters et al., 1998b). Wang et al. (1987) reported that “flanking sequences of the border repeats enhance (on the right) or attenuate (on the left) their activity”. They defined an ‘attenuation region’: a 363-bp AT-rich EcoRI/BclI fragment of the T-DNA near the LB of plasmid pTiC58. De Buck et al. (2000) stated that “it is possible that deletion of the inner border region, a piece of T-DNA present in the original Ti plasmid from which the vector was derived, causes inefficient nicking of the LB repeat, which results in read-through past the LB and the transfer of downstream-located vector sequences”. Kuraya et al. (2004) reported that “transfer of the ‘vector backbone’ from the control vectors resulted mainly from inefficient termination of formation of the transfer intermediate of the T-DNA, and additional LB sequences effectively suppressed such transfer”.

On the basis of these reports we have cloned an extra nopaline type LB together with the flanking attenuation region outside the LB of binary vector pBINAW2a, to prevent integration of backbone vector DNA in the plant genome. Ti plasmid pTiC58C1 was isolated from Agrobacterium tumefaciens strain LBA958 (kindly provided by Dr. P. Hooykaas, Leiden University, The Netherlands). The complete sequence of the T-DNA region of this Ti plasmid is published by Gielen et al. (1999). A 885-bp fragment was amplified from this Ti plasmid by PCR with primers LB2 (5′-TAACCGAGAAATGAATAAGAAG-3′ (SEQ ID NO: 54)) and LBatt (5′-GCGAGACAGATGAAACGAAGTA-3′ (SEQ ID NO: 55)). This fragment was cloned in pGEM-T and sequenced. This plasmid (pAtt1-1) was subsequently transformed to E. coli strain GM2163 (Fermentas), which is dam⁻ and dcm⁻. Plasmid DNA isolated from this strain was digested with BclI, releasing a 677-bp BclI fragment containing the Attenuation Region and the LB sequence. This fragment was cloned into BclI-digested and Alkaline Phosphatase-treated pBINAW2a plasmid DNA, isolated from an E. coli strain GM2163 transformed culture. Several colonies were analysed for the presence of the BclI fragment in the right orientation. This resulted in binary vector pBINAW5. Subsequently, a XbaI/KpnI fragment from vector pBINAW4 (see Example 2) containing the extended GBSSI promoter, GBSSI inverted repeat, and extended GBSSI terminator sequences, were cloned into the XbaI and KpnI sites of pBINAW5, resulting in binary vector pBINAW6.

Vector pBINAW6a was derived from vector pBINAW6. A double-stranded oligo with two KpnI sticky ends was ligated into the KpnI site of pBINAW6. This double-stranded oligo harbours restriction sites for AscI, SmaI and PvuI. Two orientations of the oligo are possible: one with the AscI site closest to the RB, and one with the PvuI site closest to the RB. The first orientation was selected.

pBINAW6a::R3a was developed by the cloning of a PacI/AscI genomic DNA fragment of SH23-2 into PvuI/AscI-digested pBINAW6a. SH23-2 is a subclone of the Bacterial Artificial Chromosome (BAC) SH23 (Huang et al. 2005). This BAC was partially digested with Sau3AI and the 7-10 kb fraction was ligated into the BamHI site of vector pBINPLUS. Using the restriction enzymes Pad and AscI, a 9-kb fragment (truncated SH23-2) was cut out of pBINPLUS::R3a and the fragment was ligated into the PvuI and AscI sites of pBINAW6a.

The T-DNA contains a fragment of SH23-2, in which the Coding Sequence (CDS) of the R3a gene is present. This CDS is (after transformation of the plant) regulated by the original promoter and terminator of R3a which are also present on SH23-2. The genomic fragment SH23-2 is isolated from the diploid potato clone SH83-92-488 (Huang et al., 2005).

The binary vector pBINAW6a::R3a was transformed into Agrobacterium tumefaciens strain AGL-1 by triparental mating, using helper plasmid pRK2013 (Figurski and Helinski, 1979).

### Transformation of Potato and Selection of Transformants

Internodal cuttings from in vitro grown plants of potato cultivars ‘Desiree’, ‘Aventra’ or ‘Aveka’ were used for transformation by Agrobacterium tumefaciens co-cultivation, according to the protocol described by Visser (1991).

Potato cultivar ‘Desiree’ was transformed with pBINAW6a::R3a in A. tumefaciens strain AGL-1. No selection was performed. After four weeks the first shoots were harvested and harvesting of shoots continued for about three months. No more than two regenerants per stem explant were harvested and shoots were allowed to grow on MS30 medium in a glass jars (diameter 10 cm) or plastic jars (diameter 15 cm). Each jar contained 5 cuttings. A 2 to 3 cm space was left between the cuttings and the inner wall of the jars.

### In Vitro Assay for Disease Testing

In vitro plantlets were grown at 24° C. and 16 h light/8 h dark. Plantlets with 3 to 4 fully developed leaves were used for in vitro inoculation. Depending on the potato genotype, after 2 to 4 weeks plantlet were ready for inoculation.

The 3 largest leaves of each in vitro plantlet were inoculated (1 spot per leaf) by pipetting 10 μl droplets of a zoospore suspension of 2.5×10⁴ spores/ml on the adaxial side. Inoculum preparation and inoculation were performed in sterilized conditions. Leaves touching the inner wall or the lid of the jars or the medium were excluded because we found these leaves to be more susceptible than others of the same plants. The jars with inoculated in vitro plantlets were incubated in the same climate chamber as the trays with inoculated detached leaves.

Non-transformed plantlet showing a susceptible interactions were scored as S (spreading lesion with massive sporulation) or class SQ (spreading lesion with no or little sporulation). Putative transformants showing a resistant interactions were scored as class R (no symptom or localized HR-like necrosis) or class RQ (trailing HR necrosis).

Putative transformants were transferred to new media and/or transferred to potting soil in the greenhouse.

### In Vitro Tuberization

To PCR-positive shoots 20 ml of a liquid medium was added, consisting of KI medium (‘knolinducerend’ (=tuber inducing) medium, Duchefa) with 325 g/L sucrose and 1.75 g/L CCC (chlorocholine chloride, or cyclocel.). The pots were placed in a dark growth chamber at a temperature of 18° C. After 2 to 4 weeks microtubers had developed on most shoots.

### Starch Staining

Microtubers were cut and stained with an iodine solution to assess the presence of amylose in the starch granules. Staining of the starch granules was inspected with a microscope.

### DNA Analyses

DNA was isolated from in vitro shoots by a CTAB DNA isolation protocol as described by Rogers and Bendich (1988). PCRs were performed with primers NPT1 and NPT2, trfA1 and trfA2, insB1 and insB2 (Sequence nos. 1, 2, 5, 6, 7 and 8; Wolters et al., 1998a), to study the presence of backbone vector DNA in the transformants.

Four μg of DNA of the transformants was digested with HindIII, fragments were separated by gel electrophoresis, and Southern blots were made. Blots were hybridized with an R3a probe, to check the number of T-DNA insertions.

### Border-Free Transformants

### PCR on the Border Sequences

The right border and left border sequences of the T-DNA are not derived from Solanum sp., but obtained from pBIN19 (pTiT37 borders). To analyze whether these border sequences are present or absent in the transformants PCRs were performed. For the analysis of the right border the following primers were used: primer RBcheck1 (5′-CCAATATATCCTGTCAGGTA-3′ (SEQ ID NO: 22)) and primer SHcis1 (5′-CATCATCATCCCAAGTACAA-3′ (SEQ ID NO: 5)). With these primers a fragment of 795 by was expected when DNA of vector pBINAW6a::R3a was used as template. For the analysis of the left border primer GBSSIcis1 (5′-CTCTGTCAACAGCCAAATAG-3′ (SEQ ID NO: 44)) was used in combination with primer LBcheck1 (5′-GGATATATTGTGGTGTAAAC-3′ (SEQ ID NO: 7)). With these primers a fragment of 836 by was expected when DNA of vector pBINAW6a::R3a was used as template.

### Universal Genomic Walking

To obtain T-DNA flanking sequences from the transformants the Universal GenomeWalker Kit (Clontech) was used. DNA of the transformants was digested with DraI, EcoRV, PvuII, ScaI and StuI. GenomeWalker Adaptors were ligated to the obtained fragments to create enzyme-specific libraries. To obtain RB-flanking DNA the first PCR was performed with adaptor primer AP1 (5′-GTAATACGACTCACTATAGGGC-3′ (SEQ ID NO: 8)) and primer SHGW1 (5′-TAAGTTTAATCAGAAGTTGGGTAGGAA-3′ (SEQ ID NO: 9)). The nested PCR was performed with adaptor primer AP2 (5′-ACTATAGGGCACGCGTGGT-3′ (SEQ ID NO: 10)) and primer SHGW2 (5′-GTTGGGTAGGAAGCCTGCTCTTGGAAA-3′ (SEQ ID NO: 11)). To obtain LB-flanking DNA the first PCR was performed with adaptor primer AP1 and primer GBSSIGW1 (5′-TTTACTCATCTCCTCCAGGATCCTTCT-3′ (SEQ ID NO: 45)). The nested PCR was performed with adaptor primer AP2 and primer GBSSIGW2 (5′-ATCTCCTCCAGGATCCTTCTGCTCCTC-3′ (SEQ ID NO: 46)).

### TABLES

TABLE 1
R-genes and quantitative trait loci for late blight resistance reported for
wild Solanum species

| | Locus type or | |
|---|---|---|
| Wild species | name | Chromosome |
| S. berthaultii | QTLs (4) | I, III, VII and XI |
| | Rpi-ber1 | X |
| | Rpi-ber2 | VII |
| S. bulbocastanum | RB/Rpi-blb1 | VIII |
| | Rpi-blb2 | VI |
| | Rpi-blb3 | IV |
| S. caripense | QTL (2) | unassigned |
| S. demissum | R1 | V |
| | R2 | IV |
| | R3, R6, R7 | XI |
| | R3a | XI |
| | R3b | XI |
| | R5-R11 | XI |
| | R10, R11 | XI |
| S. microdontum | QTLs (3) | IV, V and X |
| | QTL | Unassigned |
| S. mochiquense | Rpi-mcq1 (Rpi- | IX |
| | moc1) | |
| S. paucissectum | QTLs (3) | X, XI and XII |
| S. phureja | Rpi-phu1 | IX |
| S. pinnatisectum | Rpi-pnt1 | VII |
| | (Rpi1) | |
| S. vernei | QTLs (several) | VI, VIII, IX |
| Hybrids with | Rp1-abp1 | IV |
| S. tuberosum | R2-like | IV |
| | QTLs (several) | several |
| | QTLs | IV |

### REFERENCES

Bene\check{s} V, Hostomský Z, Arnold L, Pa\check{c}es V (1993) M13 and pUC vectors with new unique restriction sites for cloning. Gene 130: 151-152
De Buck S, De Wilde C, Van Montagu M, Depicker A (2000) T-DNA vector backbone sequences are frequently integrated into the genome of transgenic plants obtained by Agrobacterium-mediated transformation. Mol Breed 6: 459-468
Chambers S P, Prior S E, Barstow D A, Minton N P (1988) The pMTL nic-cloning vectors. I. Improved pUC polylinker regions to facilitate the use of sonicated DNA for nucleotide sequencing. Gene 68: 139-149
Dai W L, Deng W, Cui W Y, Zhao S Y, Wang X M (1996) Molecular cloning and sequence of potato granule-bound starch synthase gene. Acta Botanica Sinica 38: 777-784
Figurski D H, Helinski D R (1979) Replication of an origin-containing derivative of plasmid RK2 dependent on a plasmid function provided in trans. Proc Natl Acad Sci USA 76: 1648-1652
Gielen J, Terryn N, Villarroel R, Van Montagu M (1999) Complete nucleotide sequence of the T-DNA region of the plant tumour-inducing Agrobacterium tumefaciens Ti plasmid pTiC58. J Exp Bot 50: 1421-1422
Heilersig H J B, Loonen A, Bergervoet M, Wolters A M A, Visser R G F (2006) Post-transcriptional gene silencing of GBSSI in potato: effects of size and sequence of the inverted repeats. Plant Mol Biol 60: 647-662
Huang S, van der Vossen E A G, Kuang H, Vleeshouwers V G A A, Zhang N, Borm T J A, van Eck H J, Baker B, Jacobsen E, Visser R G F (2005) Comparative genomics enabled the isolation of the R3α late blight resistance gene in potato. Plant J 42: 251-261.
Kloosterman B (2006) Transcriptomic analysis of potato tuber development and tuber quality traits using microarray technology. In search of candidate genes. PhD Thesis, Wageningen University, The Netherlands. ISBN 90-8504-454-5
Kononov M E, Bassuner B, Gelvin S B (1997) Integration of T-DNA binary vector ‘backbone’ sequences into the tobacco genome: evidence for multiple complex patterns of integration. Plant J 11: 945-957
Kuraya Y, Ohta S, Fukuda M, Hiei Y, Murai N, Hamada K, Ueki J, Imaseki H, Komari T (2004) Suppression of transfer of non-T-DNA ‘vector backbone’ sequences of higher plants mediated by Agrobacterium tumefaciens. Mol Breed 14: 309-320
Ramanathan V, Veluthambi K (1995) Transfer of non-T-DNA portions of the Agrobacterium tumefaciens Ti plasmid pTiA6 from the left terminus of T_{L}-DNA. Plant Mol Biol 28: 1149-1154
Rogers S O, Bendich A J (1988) Extraction of DNA from plant tissues. In: Plant Molecular Biology Manual (Gelvin S B, Schilperoort R A, eds). Kluwer Academic Publ, Dordrecht, pp. A6/1-A6/10
Torto T. et al. (2003) EST mining and functional expression assays identify extracellular effector proteins from Phytophthorta. Genome Res. 13:1675-1685.
Van der Graaff E, den Dulk-Ras A, Hooykaas P J J (1996) Deviating T-DNA transfer from Agrobacterium tumefaciens to plants. Plant Mol Biol 31: 677-681
Van der Leij F R, Visser R G F, Ponstein A S, Jacobsen E, Feenstra W J (1991a) Sequence of the structural gene for granule-bound starch synthase of potato (Solanum tuberosum L.) and evidence for a single point deletion in the amf allele. Mol Gen Gent 228: 240-248
Van der Leij F R, Visser R G F, Oosterhaven K, van der Kop D A M, Jacobsen E, Feenstra W J (1991b) Complementation of the amylose-free starch mutant of potato (Solanum tuberosum) by the gene encoding granule-bound starch synthase. Theor Appl Genet 82: 289-295
Van der Leij F R, Abeln E C A, Hesseling-Meinders A, Feenstra W J (1993) A putative β-glucanase pseudogene behind the potato GBSS gene. Plant Mol Biol 21: 567-571
Van der Vossen E, Sikkema A, to Lintel Hekkert B, Gros J, Stevens P, Muskens M, Wouters D, Pereira A, Stiekema W, Allefs S (2003) An ancient R gene from the wild potato species Solanum bulbocastanum confers broad-spectrum resistance to Phytophthora infestans in cultivated potato and tomato. Plant J 36: 867-882
Visser R G F, Somhorst I, Kuipers G J, Ruys N J, Feenstra W J, Jacobsen E (1991a) Inhibition of the expression of the gene for granule-bound starch synthase in potato by antisense constructs. Mol Gen Genet 225: 289-296
Visser R G F, Stolte A, Jacobsen E (1991b) Expression of a chimaeric granule-bound starch synthase-GUS gene in transgenic potato plants. Plant Mol Biol 17: 691-699
Wang K, Genetello C, Van Montagu M, Zambryski P C (1987) Sequence context of the T-DNA border repeat element determines its relative activity during T-DNA transfer to plant cells. Mol Gen Gent 210: 338-346
Wenck A, Czakó M, Kanevski I, Márton L (1997) Frequent collinear long transfer of DNA inclusive of the whole binary vector during Agrobacterium-mediated transformation. Plant Mol Biol 34: 913-922
Windels P, De Buck S, Van Bockstaele E, De Loose M, Depicker A (2003) T-DNA integration in Arabidopsis chromosomes. Presence and orgin of filler DNA sequences. Plant Physiol 133: 2061-2068
Wolters A M A, Janssen E M, Rozeboom-Schippers M G M, Jacobsen E, Visser R G F (1998a) Composition of endogenous alleles can influence the level of antisense inhibition of granule-bound starch synthase gene expression in tetraploid potato plants. Mol Breeding 4: 343-358
Wolters A M A, Trindade L M, Jacobsen E, Visser R G F (1998b) Fluorescence in situ hybridization on extended DNA fibres as a tool to analyse complex T-DNA loci in potato. Plant J 13: 837-847
Zhu Q H, Ramm K, Eamens A L, Dennis E S, Upadhyaya N M (2006) Transgene structures suggest that multiple mechanisms are involved in T-DNA integration in plants. Plant Science 171: 308-322

### DESCRIPTION OF FIGURES

FIG. 1. Nucleotide sequence of clone Blb25-B2 (8461 bp) (SEQ ID NO: 56) containing the Rpi-blb3 gene and regulatory sequences. The Rpi-blb3 coding region of 2544 by is highlighted in lower case (2944-5487). The upstream 2732 nt (211-2942) and the downstream 882 nt (5488-6370) harbour the regulatory sequences.

FIG. 2. Deduced Rpi-blb3 protein sequence (SEQ ID NO: 57). The amino-acid sequence deduced from the DNA sequence of Rpi-blb3 is divided into three domains (CC, NB-ARC and LRR).

Hydrophobic residues in the CC domain are underlined. Conserved motifs in R proteins are written in italic in the NBS domain. Residues matching the consensus of the cytoplasmic LRR are indicated in bold in the LRR domain.

FIG. 3. shows the StTLRP delta 7 sequences; the intron and the 3′UTR are underlined.

FIG. 4. Sequence alignment of Rpi-sto1, Rpi-pta1 and Rpi-blb1. A. DNA sequence alignment. B. Amino acid sequence alignment.

FIG. 5. Binary vector pBINAW4, containing the GBSS inverted repeat cDNA.

FIG. 6. Binary vector pBINAW4a::R3a containing the GBSS inverted repeat cDNA and the Phytophthora infestans resistance gene R3a.

FIG. 7. Analysis of the integrated T-DNA borders in the single copy, marker-free R3a Desiree transformants.

The right and left border integration of the T-DNA of 8 independent integration events into the potato genome are shown. The first line shows the original T-DNA sequence, the other lines show the sequences found in the different transgenic clones. The nucleotide sequence belonging to the 25 bp right (A) and left (B) border repeat are shaded, and the “/” indicates the positions of the T-DNA nicking. The remaining nucleotide sequences shown are part of the genomic sequence of R3a of the T-DNA.

FIG. 8. Binary vector pBINAW2b::R3a with reduced T-DNA borders

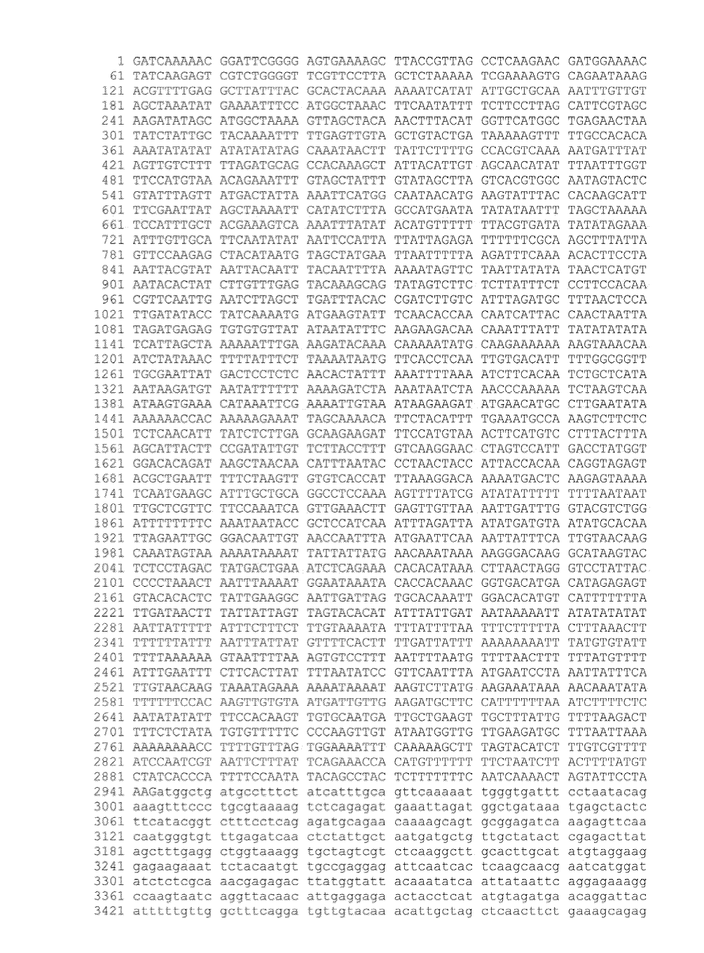

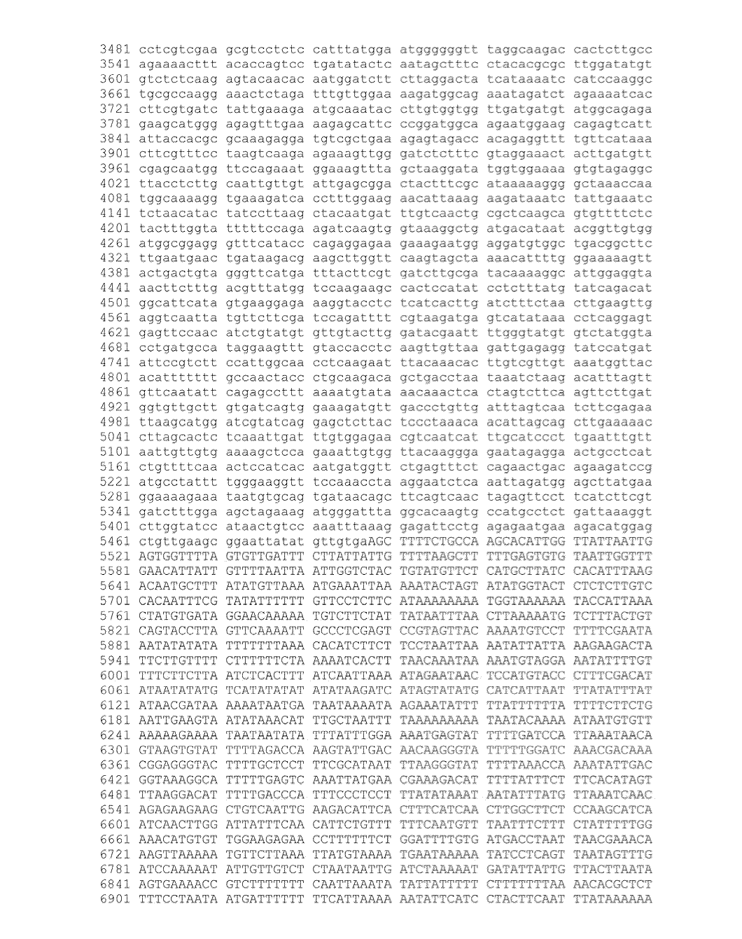

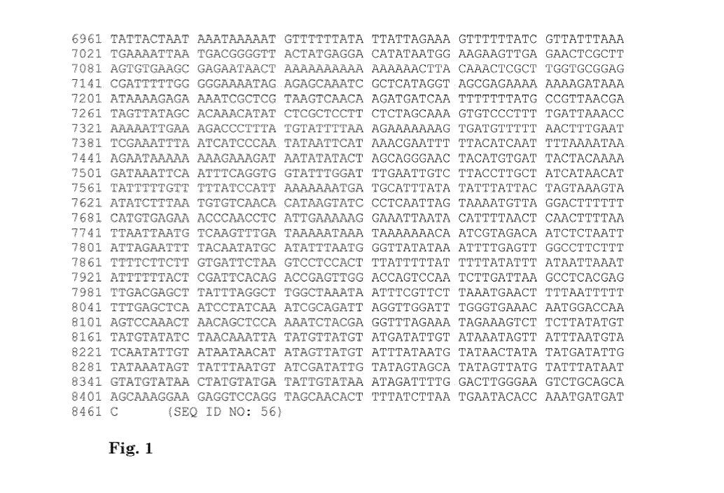

### INCORPORATION OF SEQUENCE LISTING

Incorporated herein by reference in its entirety is the Sequence Listing for the application. The Sequence Listing is disclosed on a computer-readable ASCII text file titled, “Sequencelisting19JAN2010_{—}294-362.txt”, created on Jan. 19, 2010. The sequence.txt file is 111 kb in size.

1. A method for obtaining a plant that has been provided with a nucleic acid of interest comprising providing
a recombinant nucleic acid comprising said nucleic acid of interest
transferring said recombinant nucleic acid to a plant cell
producing a plant from said cell
and determining the presence of said nucleic acid of interest and the absence of vector backbone and/or border sequences,
wherein said nucleic acid of interest is essentially a plant nucleic acid.

2. A method according to claim 1, wherein said nucleic acid of interest is a cDNA sequence.

3. A method according to claim 1, wherein said nucleic acid of interest is an inverted (repeat) sequence.

4. A method according to claim 1, wherein said nucleic acid of interest is a genomic sequence.

5. A method according to claim 1, wherein said nucleic acid of interest and said plant are from the same crossable species.

6. A method according to claim 1, wherein said plant nucleic acid is a Solanaceae nucleic acid.

7. A method according to claim 1, wherein said plant nucleic acid is an open reading frame that is under control of native 5′ and 3′ nucleic acid sequences.

8. A method according to claim 1, wherein said nucleic acid of interest is a functional R-gene.

9. A method according to claim 1, further comprising exposing the resulting plant to at least one oomycte elicitor and determining the presence or absence of a reaction to said at least one elicitor.

10. A method according to claim 9, wherein said elicitor is an elicitor from P. infestans.

11. A method according to claim 9, wherein said reaction is a hypersensitive response (HR).

12. A method according to claim 1, wherein said recombinant nucleic acid is integrated into the genome of said plant.

13. A method according to claim 1, wherein said functional R-gene is a gene encoding Rpi-blb3 or a gene encoding a functional fragment thereof or a gene encoding a derivative thereof.

14. A method according to claim 1, wherein said functional R-gene is a gene encoding Rpi-sto1 or a gene encoding a functional fragment thereof or a gene encoding a derivative thereof.

15. A method according to claim 1, wherein said functional R-gene is a gene encoding Rpi-pta1 or a gene encoding a functional fragment thereof or a gene encoding a derivative thereof.

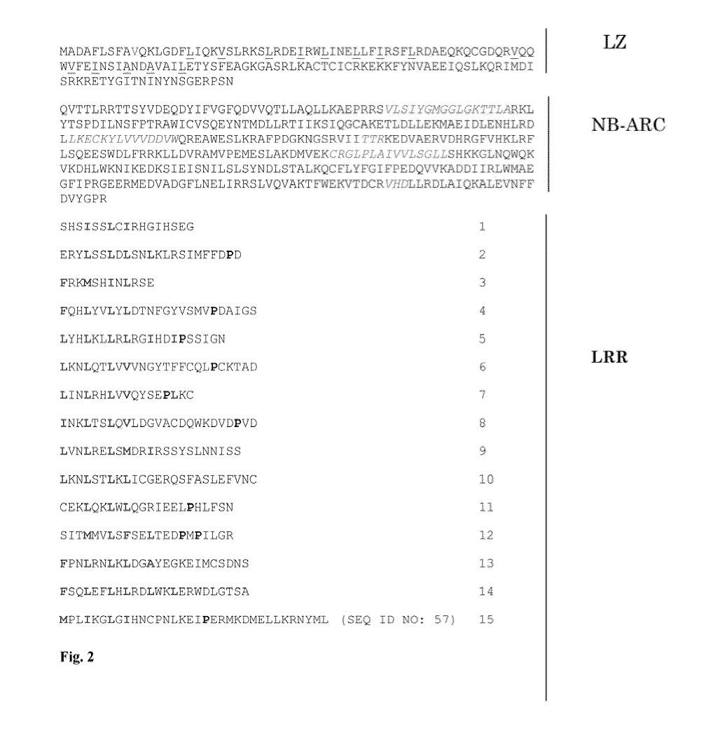

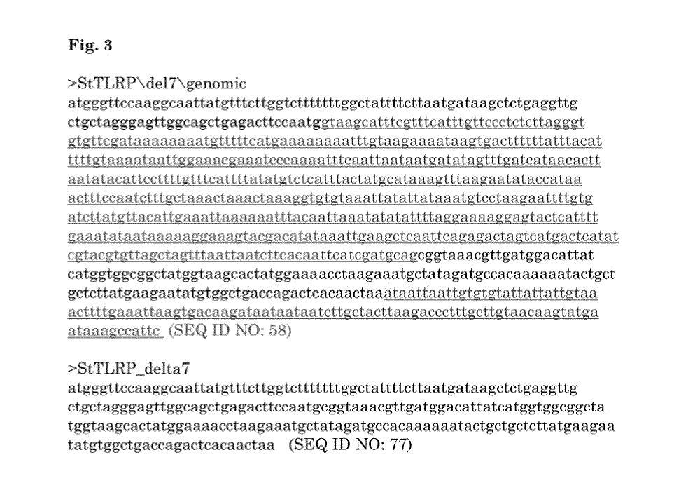

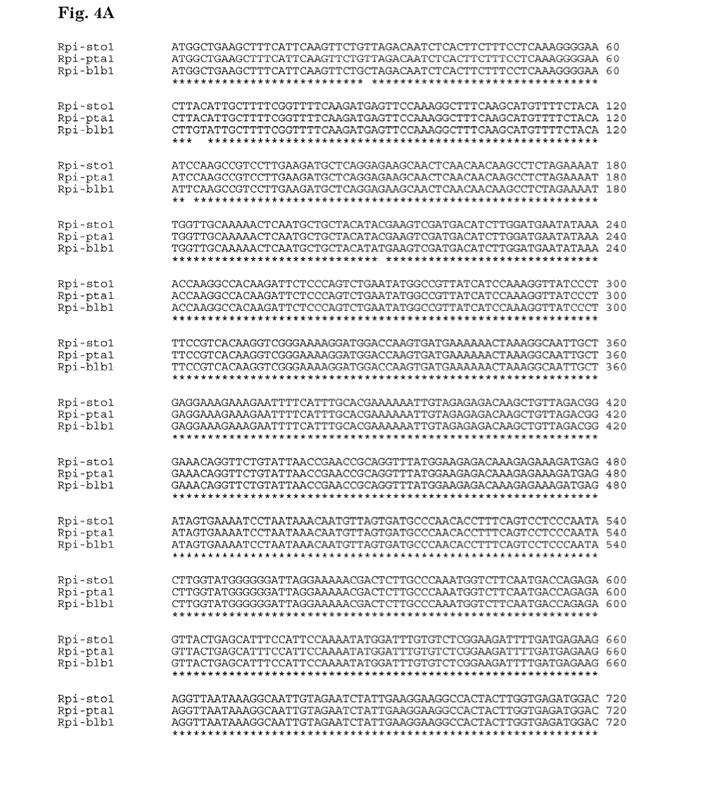

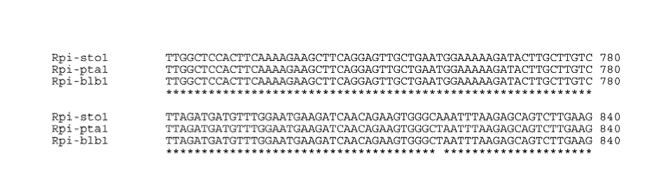

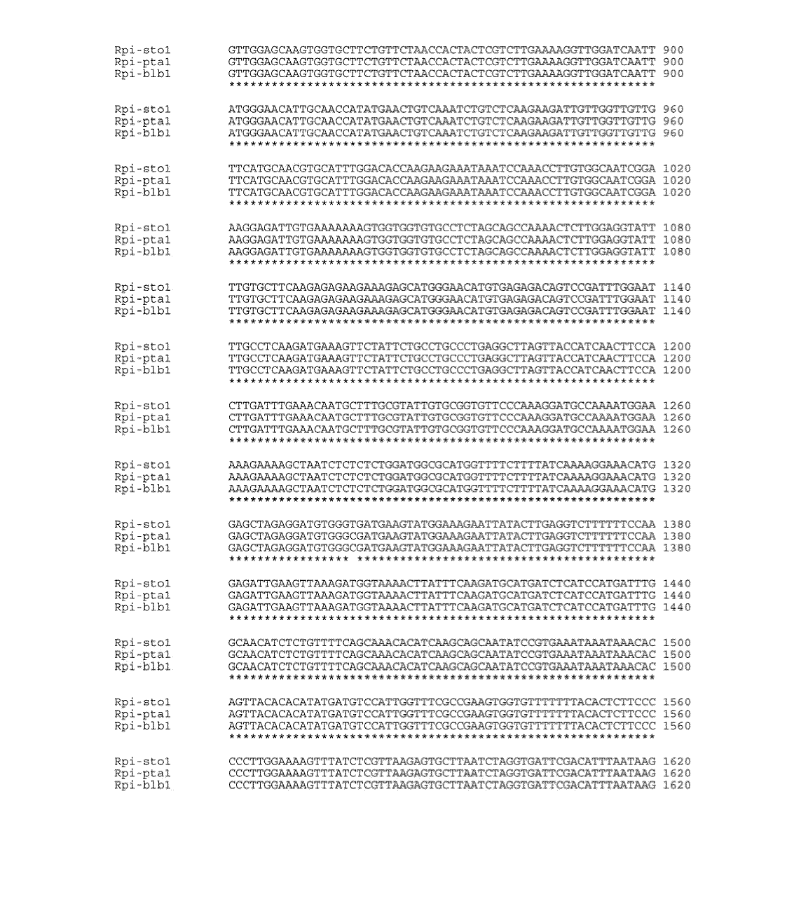

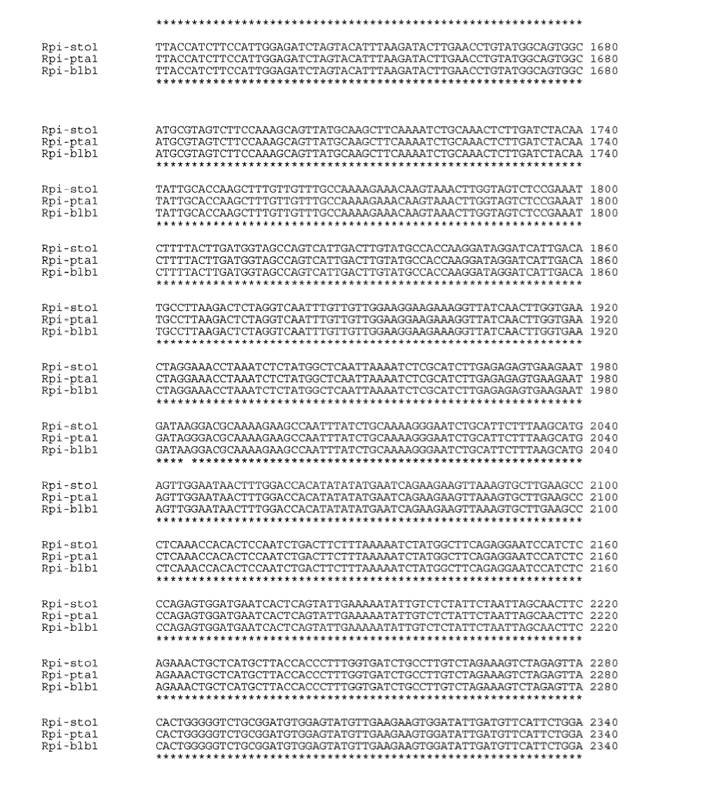

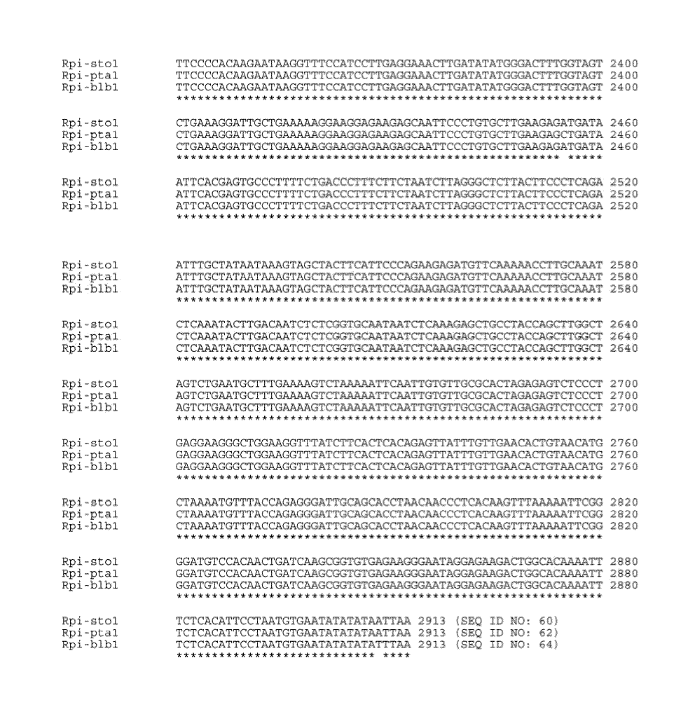

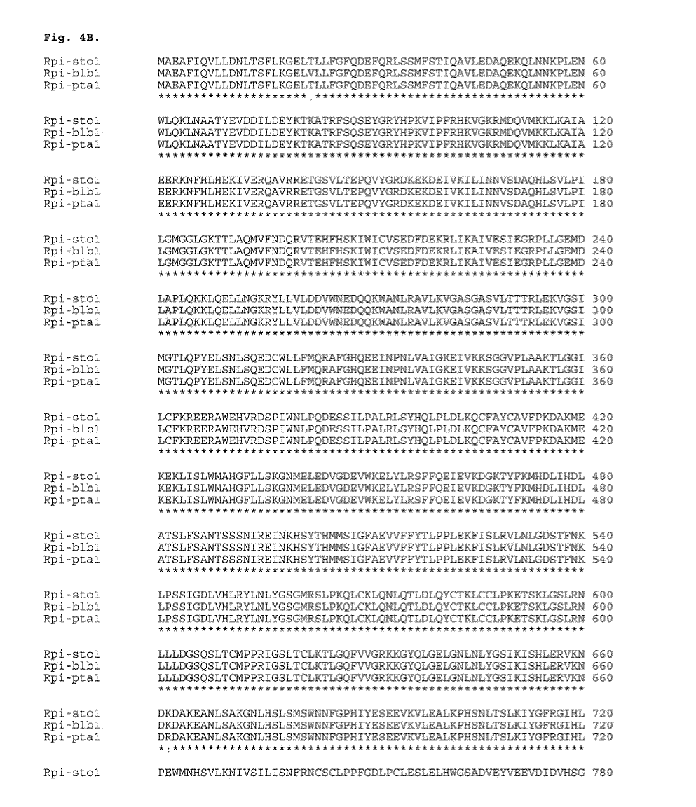

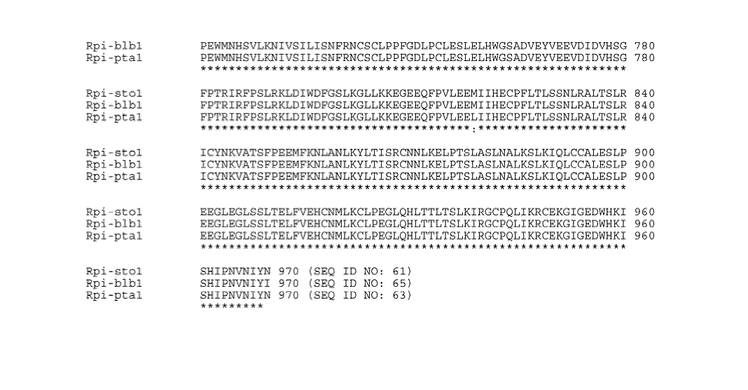

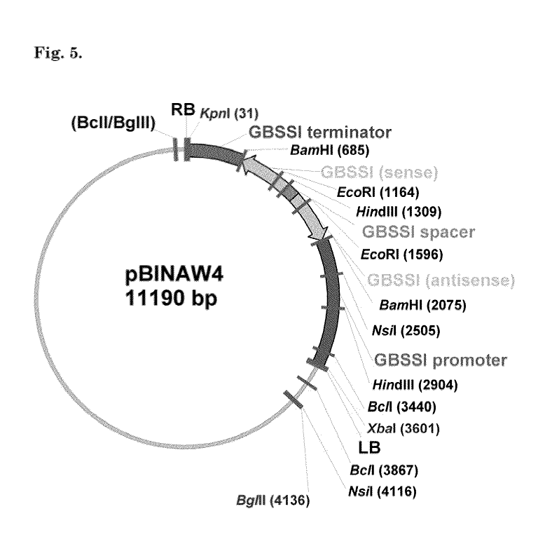

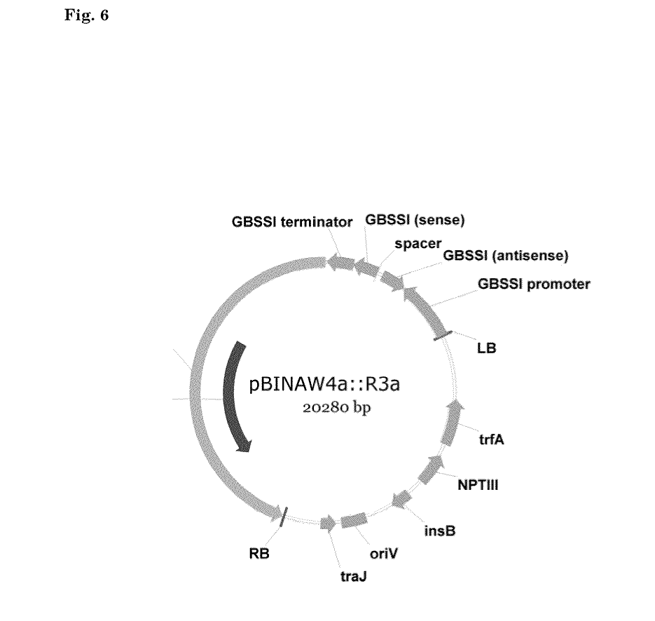

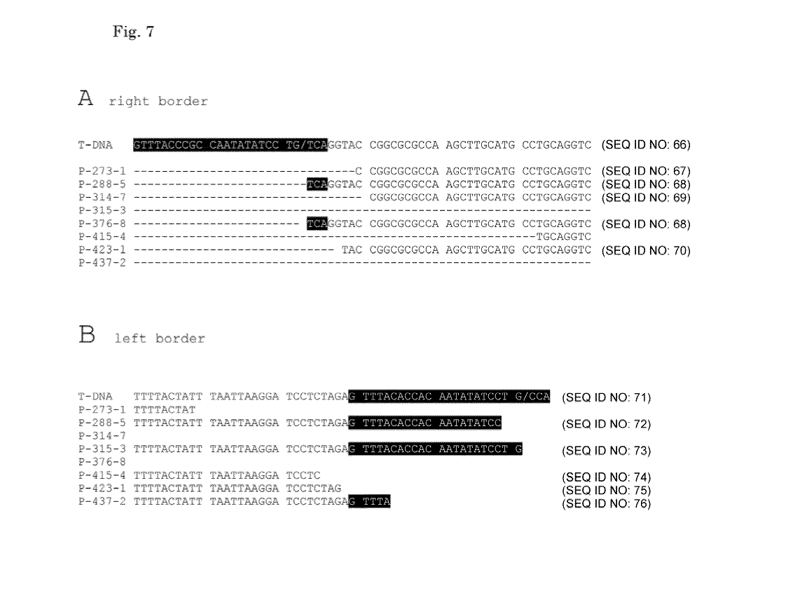

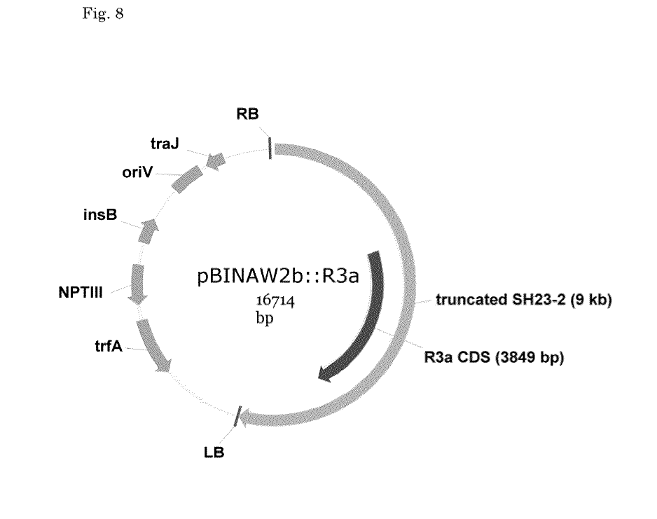

16. A method according to claim 12, wherein said functional R-gene is a gene encoding Rpi-blb-3 as depicted in FIG. 2.

17. A plant obtainable by a method according to claim 1.

18. An, Agrobacterium transformed plant free from non-Solanum T-DNA border sequences carrying a heterologous, cisgenic gene of interest.

19. A plant according to claim 17, wherein said gene of interest is under control of native 5′ and 3′ sequences.

20. A plant according to claim 17, wherein said gene of interest is a genomic sequence.

21. A plant according to claim 17, wherein said gene of interest is from the same crossable species.


SEQUENCE LISTING (77 sequences)

SEQ ID NO: 1 - protein, 5 aa, Artificial Sequence
  Synthetic amino acid sequence
RXALR

SEQ ID NO: 2 - DNA, 26 bp, Artificial Sequence
  Synthetic nucleotide sequence primer
agcttggcgcgcccgggttaattaag

SEQ ID NO: 3 - DNA, 26 bp, Artificial Sequence
  Synthetic nucleotide sequence primer
aattcttaattaacccgggcgcgcca

SEQ ID NO: 4 - DNA, 20 bp, Artificial Sequence
  Synthetic nucleotide sequence primer
ccaatatatcctgtcaaaca

SEQ ID NO: 5 - DNA, 20 bp, Artificial Sequence
  Synthetic nucleotide sequence primer
catcatcatcccaagtacaa

SEQ ID NO: 6 - DNA, 20 bp, Artificial Sequence
  Synthetic nucleotide sequence primer
aagggcaaacataaccattc

SEQ ID NO: 7 - DNA, 20 bp, Artificial Sequence
  Synthetic nucleotide sequence primer
ggatatattgtggtgtaaac

SEQ ID NO: 8 - DNA, 22 bp, Artificial Sequence
  Synthetic nucleotide sequence primer
gtaatacgactcactatagggc

SEQ ID NO: 9 - DNA, 27 bp, Artificial Sequence
  Synthetic nucleotide sequence primer
taagtttaatcagaagttgggtaggaa

SEQ ID NO: 10 - DNA, 19 bp, Artificial Sequence
  Synthetic nucleotide sequence primer
actatagggcacgcgtggt

SEQ ID NO: 11 - DNA, 27 bp, Artificial Sequence
  Synthetic nucleotide sequence primer
gttgggtaggaagcctgctcttggaaa

SEQ ID NO: 12 - DNA, 27 bp, Artificial Sequence
  Synthetic nucleotide sequence primer
gtatgtatgtgtagttaatggggtagt

SEQ ID NO: 13 - DNA, 27 bp, Artificial Sequence
  Synthetic nucleotide sequence primer
acggtttctaaattaacgtagccaata

SEQ ID NO: 14 - DNA, 21 bp, Artificial Sequence
  Synthetic nucleotide sequence primer
gcggtcctgatcaatcgtcac

SEQ ID NO: 15 - DNA, 30 bp, Artificial Sequence
  Synthetic nucleotide sequence primer
ggtacctgacaggatatattggcgggtaaa

SEQ ID NO: 16 - DNA, 32 bp, Artificial Sequence
  Synthetic nucleotide sequence primer
ggtacctctagagtttacaccacaatatatcc

SEQ ID NO: 17 - DNA, 22 bp, Artificial Sequence
  Synthetic nucleotide sequence primer
gcgggtttaacctacttccttt

SEQ ID NO: 18 - DNA, 22 bp, Artificial Sequence
  Synthetic nucleotide sequence primer
cggcgcgcccgggttaattaag

SEQ ID NO: 19 - DNA, 30 bp, Artificial Sequence
  Synthetic nucleotide sequence primer
gatccttaattaacccgggcgcgccggtac

SEQ ID NO: 20 - DNA, 23 bp, Artificial Sequence
  Synthetic nucleotide sequence primer AL79
gagaatggaagatttgggtgaag

SEQ ID NO: 21 - DNA, 23 bp, Artificial Sequence
  Synthetic nucleotide sequence primer AL80
ctaatctcaccagttggctgttc

SEQ ID NO: 22 - DNA, 20 bp, Artificial Sequence
  Synthetic nucleotide sequence primer
ccaatatatcctgtcaggta

SEQ ID NO: 23 - DNA, 22 bp, Artificial Sequence
  Synthetic nucleotide sequence primer
agctgtgaaggcaaagatgagg

SEQ ID NO: 24 - DNA, 22 bp, Artificial Sequence
  Synthetic nucleotide sequence primer
cgtcactgccatcatggtagat

SEQ ID NO: 25 - DNA, 21 bp, Artificial Sequence
  Synthetic nucleotide sequence primer
taccgctaccacttgacattc

SEQ ID NO: 26 - DNA, 21 bp, Artificial Sequence
  Synthetic nucleotide sequence primer
gcaccccaggctttacacttt

SEQ ID NO: 27 - DNA, 27 bp, Artificial Sequence
  Synthetic nucleotide sequence primer
ctctagaagttcgagacactggctacg

SEQ ID NO: 28 - DNA, 28 bp, Artificial Sequence
  Synthetic nucleotide sequence primer
ggatcctggaggagatgagtaaaagtta

SEQ ID NO: 29 - DNA, 27 bp, Artificial Sequence
  Synthetic nucleotide sequence primer
ggatccaaacgtatttactagcgaact

SEQ ID NO: 30 - DNA, 26 bp, Artificial Sequence
  Synthetic nucleotide sequence primer
ggtaccaaagagacaggtgccgttat

SEQ ID NO: 31 - DNA, 23 bp, Artificial Sequence
  Synthetic nucleotide sequence primer AMYLM F
agataagctttctcattccttgc

SEQ ID NO: 32 - DNA, 19 bp, Artificial Sequence
  Synthetic nucleotide sequence primer
tcctccaggatccttctgg

SEQ ID NO: 33 - DNA, 20 bp, Artificial Sequence
  Synthetic nucleotide sequence primer
tcaggtaccaaagagacagg

SEQ ID NO: 34 - DNA, 20 bp, Artificial Sequence
  Synthetic nucleotide sequence primer
ggagcagaaggatccaaacg

SEQ ID NO: 35 - DNA, 18 bp, Artificial Sequence
  Synthetic nucleotide sequence primer
tgccgttatgtaaaggag

SEQ ID NO: 36 - DNA, 22 bp, Artificial Sequence
  Synthetic nucleotide sequence primer
agcttctttcatatgaccaacc

SEQ ID NO: 37 - DNA, 24 bp, Artificial Sequence
  Synthetic nucleotide sequence primer
caggatatattgtggtgtaaactc

SEQ ID NO: 38 - DNA, 20 bp, Artificial Sequence
  Synthetic nucleotide sequence primer
tatctttgctcaggaccctg

SEQ ID NO: 39 - DNA, 18 bp, Artificial Sequence
  Synthetic nucleotide sequence primer
gcagaaggatcctggagg

SEQ ID NO: 40 - DNA, 19 bp, Artificial Sequence
  Synthetic nucleotide sequence primer
aactcgaagtcagcctgcg

SEQ ID NO: 41 - DNA, 21 bp, Artificial Sequence
  Synthetic nucleotide sequence primer
accgctaccacttgacattcc

SEQ ID NO: 42 - DNA, 23 bp, Artificial Sequence
  Synthetic nucleotide sequence primer
caaatgcaacagtatcttgtacc

SEQ ID NO: 43 - DNA, 23 bp, Artificial Sequence
  Synthetic nucleotide sequence primer
tcaatgtttgttacatttcttcc

SEQ ID NO: 44 - DNA, 20 bp, Artificial Sequence
  Synthetic nucleotide sequence primer
ctctgtcaacagccaaatag

SEQ ID NO: 45 - DNA, 27 bp, Artificial Sequence
  Synthetic nucleotide sequence primer
tttactcatctcctccaggatccttct

SEQ ID NO: 46 - DNA, 27 bp, Artificial Sequence
  Synthetic nucleotide sequence primer
atctcctccaggatccttctgctcctc

SEQ ID NO: 47 - DNA, 20 bp, Artificial Sequence
  Synthetic nucleotide sequence primer
cgctttcagaatctattact

SEQ ID NO: 48 - DNA, 27 bp, Artificial Sequence
  Synthetic nucleotide sequence primer
atttcatgcgcatattcccgatcaaac

SEQ ID NO: 49 - DNA, 27 bp, Artificial Sequence
  Synthetic nucleotide sequence primer
tcccgatcaaacttaaattactagact

SEQ ID NO: 50 - DNA, 29 bp, Artificial Sequence
  Synthetic nucleotide sequence primer
ggatccatgggttccaaggcaattatgtt

SEQ ID NO: 51 - DNA, 29 bp, Artificial Sequence
  Synthetic nucleotide sequence primer
ggatccgaatggctttattcatacttgtt

SEQ ID NO: 52 - DNA, 27 bp, Artificial Sequence
  Synthetic nucleotide sequence primer
attgccttggaacccatggatccttct

SEQ ID NO: 53 - DNA, 27 bp, Artificial Sequence
  Synthetic nucleotide sequence primer
tggaacccatggatccttctgctcctc

SEQ ID NO: 54 - DNA, 22 bp, Artificial Sequence
  Synthetic nucleotide sequence primer
taagcgagaaatgaataagaag

SEQ ID NO: 55 - DNA, 22 bp, Artificial Sequence
  Synthetic nucleotide sequence primer
gcgagacagatgaaacgaagta

SEQ ID NO: 56 - DNA, 8461 bp, Solanum bulbocastanum
  CDS (2944)..(5487)
gatcaaaaacggattcggggagtgaaaagcttaccgttagcctcaagaacgatggaaaactatcaagagtcgtctggggttcgttccttagctctaaaaatcgaaaagtgcagaataaagacgttttgaggcttatttacgcactacaaaaaaatcatatattgctgcaaaatttgttgtagctaaatatgaaaatttccatggctaaacttcaatattttcttccttagcattcgtagcaagatatagcatggctaaaagttagctacaaactttacatggttcatggctgagaactaatatctattgctacaaaatttttgagttgtagctgtactgataaaaagtttttgccacacaaaatatatatatatatatagcaaataactttattcttttgccacgtcaaaaatgatttatagttgtctttttagatgcagccacaaagctattacattgtagcaacatatttaatttggtttccatgtaaacagaaatttgtagctatttgtatagcttagtcacgtggcaatagtactcgtatttagttatgactattaaaattcatggcaataacatgaagtatttaccacaagcattttcgaattatagctaaaattcatatctttagccatgaatatatataattttagctaaaaatccatttgctacgaaagtcaaaatttatatacatgtttttttacgtgatatatatagaaaatttgttgcattcaatatataattccattattattagagattttttcgcaagctttattagttccaagagctacataatgtagctatgaattaatttttaagatttcaaaacacttcctaaattacgtataattacaatttacaattttaaaaatagttctaattatatataactcatgtaatacactatcttgtttgagtacaaagcagtatagtcttctcttatttctccttccacaacgttcaattgaatcttagcttgatttacaccgatcttgtcatttagatgctttaactccattgatatacctatcaaaatgatgaagtatttcaacaccaacaatcattaccaactaattatagatgagagtgtgtgttatataatatttcaagaagacaacaaatttatttatatatatatcattagctaaaaaatttgaaagatacaaacaaaaatatgcaagaaaaaaaagtaaacaaatctataaacttttatttcttaaaataatgttcacctcaattgtgacatttttggcggtttgcgaattatgactcctctcaacactatttaaattttaaaatcttcacaatctgctcataaataagatgtaatattttttaaaagatctaaaataatctaaacccaaaaatctaagtcaaataagtgaaacataaattcgaaaattgtaaataagaagatatgaacatgccttgaatataaaaaaaccacaaaaagaaattagcaaaacattctacattttgaaatgccaaagtcttctctctcaacatttatctcttgagcaagaagatttccatgtaaacttcatgtcctttactttaagcattacttccgatattgttcttacctttgtcaaggaacctagtccattgacctatggtggacacagataagctaacaacatttaataccctaactaccattaccacaacaggtagagtacgctgaatttttctaagttgtgtcaccatttaaaggacaaaaatgactcaagagtaaaatcaatgaagcatttgctgcaggcctccaaaagttttatcgatatatttttttttaataatttgctcgttcttccaaatcagttgaaacttgagttgttaaaattgatttggtacgtctggattttttttcaaataataccgctccatcaaatttagattaatatgatgtaatatgcacaattagaattgcggacaattgtaaccaatttaatgaattcaaaattatttcattgtaacaagcaaatagtaaaaaataaaattattattatgaacaaataaaaagggacaaggcataagtactctcctagactatgactgaaatctcagaaacacacataaacttaactagggtcctattaccccctaaactaatttaaaatggaataaatacaccacaaacggtgacatgacatagagagtgtacacactctattgaaggcaattgattagtgcacaaattggacacatgtcatttttttattgataactttattattagttagtacacatatttattgataataaaaattatatatatataattatttttatttctttctttgtaaaatatttattttaatttctttttactttaaacttttttttatttaatttattatgttttcacttttgattatttaaaaaaaatttatgtgtattttttaaaaaagtaattttaaagtgtcctttaattttaatgttttaacttttttatgttttatttgaatttcttcacttattttaatatccgttcaatttaatgaatcctaaattatttcattgtaacaagtaaatagaaaaaaataaaataagtcttatgaagaaataaaaacaaatatattttttccacaagttgtgtaatgattgttgaagatgcttccattttttaaatcttttctcaatatatattttccacaagttgtgcaatgattgctgaagttgctttattgttttaagacttttctctatatgtgtttttccccaagttgtataatggttgttgaagatgctttaattaaaaaaaaaaaccttttgtttagtggaaaatttcaaaaagctttagtacatctttgtcgttttatccaatcgtaattctttattcagaaaccacatgttttttttctaatcttacttttatgtctatcacccattttccaatatacagcctactctttttttcaatcaaaactagtattcctaaagatggctgatgcctttctatcatttgcagttcaaaaattgggtgatttcctaatacagaaagtttccctgcgtaaaagtctcagagatgaaattagatggctgataaatgagctactcttcatacggtctttcctcagagatgcagaacaaaagcagtgcggagatcaaagagttcaacaatgggtgtttgagatcaactctattgctaatgatgctgttgctatactcgagacttatagctttgaggctggtaaaggtgctagtcgtctcaaggcttgcacttgcatatgtaggaaggagaagaaattctacaatgttgccgaggagattcaatcactcaagcaacgaatcatggatatctctcgcaaacgagagacttatggtattacaaatatcaattataattcaggagaaaggccaagtaatcaggttacaacattgaggagaactacctcatatgtagatgaacaggattacatttttgttggctttcaggatgttgtacaaacattgctagctcaacttctgaaagcagagcctcgtcgaagcgtcctctccatttatggaatggggggtttaggcaagaccactcttgccagaaaactttacaccagtcctgatatactcaatagctttcctacacgcgcttggatatgtgtctctcaagagtacaacacaatggatcttcttaggactatcataaaatccatccaaggctgcgccaaggaaactctagatttgttggaaaagatggcagaaatagatctagaaaatcaccttcgtgatctattgaaagaatgcaaataccttgtggtggttgatgatgtatggcagagagaagcatgggagagtttgaaaagagcattcccggatggcaagaatggaagcagagtcattattaccacgcgcaaagaggatgtcgctgaaagagtagaccacagaggttttgttcataaacttcgtttcctaagtcaagaagaaagttgggatctctttcgtaggaaactacttgatgttcgagcaatggttccagaaatggaaagtttagctaaggatatggtggaaaagtgtagaggcttacctcttgcaattgttgtattgagcggactactttcgcataaaaaggggctaaaccaatggcaaaaggtgaaagatcacctttggaagaacattaaagaagataaatctattgaaatctctaacatactatccttaagctacaatgatttgtcaactgcgctcaagcagtgttttctctactttggtatttttccagaagatcaagtggtaaaggctgatgacataatacggttgtggatggcggagggtttcatacccagaggagaagaaagaatggaggatgtggctgacggcttcttgaatgaactgataagacgaagcttggttcaagtagctaaaacattttgggaaaaagttactgactgtagggttcatgatttacttcgtgatcttgcgatacaaaaggcattggaggtaaacttctttgacgtttatggtccaagaagccactccatatcctctttatgtatcagacatggcattcatagtgaaggagaaaggtacctctcatcacttgatctttctaacttgaagttgaggtcaattatgttcttcgatccagattttcgtaagatgagtcatataaacctcaggagtgagttccaacatctgtatgtgttgtacttggatacgaattttgggtatgtgtctatggtacctgatgccataggaagtttgtaccacctcaagttgttaagattgagaggtatccatgatattccgtcttccattggcaacctcaagaatttacaaacacttgtcgttgtaaatggttacacatttttttgccaactaccctgcaagacagctgacctaataaatctaagacatttagttgttcaatattcagagcctttaaaatgtataaacaaactcactagtcttcaagttcttgatggtgttgcttgtgatcagtggaaagatgttgaccctgttgatttagtcaatcttcgagaattaagcatggatcgtatcaggagctcttactccctaaacaacattagcagcttgaaaaaccttagcactctcaaattgatttgtggagaacgtcaatcatttgcatcccttgaatttgttaattgttgtgaaaagctccagaaattgtggttacaagggagaatagaggaactgcctcatctgttttcaaactccatcacaatgatggttctgagtttctcagaactgacagaagatccgatgcctattttgggaaggtttccaaacctaaggaatctcaaattagatggagcttatgaaggaaaagaaataatgtgcagtgataacagcttcagtcaactagagttccttcatcttcgtgatctttggaagctagaaagatgggatttaggcacaagtgccatgcctctgattaaaggtcttggtatccataactgtccaaatttaaaggagattcctgagagaatgaaagacatggagctgttgaagcggaattatatgttgtgaagcttttctgccaagcacattggttattaattgagtggttttagtgttgatttcttattattgttttaagctttttgagtgtgtaattggtttgaacattattgttttaattaattggtctactgtatgttctcatgcttatccacatttaagacaatgctttatatgttaaaatgaaattaaaaatactagtatatggtactctctcttgtccacaatttcgtatattttttgttcctcttcataaaaaaaatggtaaaaaataccattaaactatgtgataggaacaaaaatgtcttctattataatttaacttaaaaatgtctttactgtcagtaccttagttcaaaattgccctcgagtccgtagttacaaaatgtcctttttcgaataaatatatatatttttttaaacacatcttcttcctaattaaaatattattaaagaagactattcttgttttcttttttctaaaaatcactttaacaaataaaaatgtaggaaatattttgttttcttcttaatctcactttatcaattaaaatagaataactccatgtaccctttcgacatataatatatgtcatatatatatataagatcatagtatatgcatcattaatttatatttatataacgataaaaaataatgataataaaataagaaatatttttattttttattttcttctgaattgaagtaatataaacatttgctaattttaaaaaaaaataatacaaaaataatgtgttaaaaagaaaataataatatatttatttggaaaatgagtatttttgatccattaaataacagtaagtgtatttttagaccaaagtattgacaacaagggtatttttggatcaaacgacaaacggagggtacttttgctcctttcgcataatttaagggtatttttaaaccaaaatattgacggtaaaggcatttttgagtcaaattatgaacgaaagacatttttatttctttcacatagtttaaggacatttttgacccatttccctcctttatataaataatatttatgttaaatcaacagagaagaagctgtcaattgaagacattcactttcatcaacttggcttctccaagcatcaatcaacttggattatttcaacattctgttttttcaatgtttaatttctttctatttttggaaacatgtgttggaagagaaccttttttctggattttgtgatgacctaattaacgaaacaaagttaaaaatgttcttaaattatgtaaaatgaataaaaatatcctcagttaatagtttgatccaaaaatattgttgtctctaataattgatctaaaaatgatattattgttacttaataagtgaaaaccgtctttttttcaattaaatatattatttttctttttttaaaacacgctcttttcctaataatgattttttttcattaaaaaatattcatcctacttcaatttataaaaaatattactaataaataaaaatgttttttatattattagaaagttttttatcgttatttaaatgaaaattaatgacggggttactatgaggacatataatggaagaagttgagaactcgcttagtgtgaagcgagaataactaaaaaaaaaaaaaaaacttacaaactcgcttggtgcggagcgatttttgggggaaaatagagagcaaatcgctcataggtagcgagaaaaaaaagataaaataaaagagaaaatcgctcgtaagtcaacaagatgatcaatttttttatgccgttaacgatagttatagcacaaacatatctcgctccttctctagcaaagtgtcccttttgattaaaccaaaaattgaaagaccctttatgtattttaaagaaaaaaagtgatgtttttaactttgaattcgaaatttaatcatcccaatataattcataaacgaatttttacatcaattttaaaataaagaataaaaaaaagaaagataatatatactagcagggaactacatgtgattactacaaaagataaattcaatttcaggtggtatttggatttgaattgtcttaccttgctatcataacattatttttgtttttatccattaaaaaaatgatgcatttatatatttattactagtaaagtaatatctttaatgtgtcaacacataagtatccctcaattagtaaaatgttaggacttttttcatgtgagaaacccaacctcattgaaaaaggaaattaatacattttaactcaacttttaattaattaatgtcaagtttgataaaaataaataaaaaaacaatcgtagacaatctctaattattagaattttacaatatgcatatttaatgggttatataaattttgagttggccttctttttttcttcttgtgattctaagtcctccactttatttttatttttatatttataattaaatattttttactcgattcacagaccgagttggaccagtccaatcttgattaagcctcacgagttgacgagcttatttaggcttggctaaataatttcgttcttaaatgaacttttaattttttttgagctcaatcctatcaaatcgcagattaggttggatttgggtgaaacaatggaccaaagtccaaactaacagctccaaaatctacgaggtttagaaatagaaagtcttcttatatgttatgtatatctaacaaattatatgttatgtatgatattgtataaatagttatttaatgtatcaatattgtataataacatatagttatgtatttataatgtataactatatatgatattgtataaatagttatttaatgtatcgatattgtatagtagcatatagttatgtatttataatgtatgtataactatgtatgatattgtataaatagattttggacttgggaagtctgcagcaagcaaaggaagaggtccaggtagcaacacttttatcttaatgaatacaccaaatgatgatc

SEQ ID NO: 57 - protein, 847 aa, Solanum bulbocastanum
MADAFLSFAVQKLGDFLIQKVSLRKSLRDEIRWLINELLFIRSFLRDAEQKQCGDQRVQQWVFEINSIANDAVAILETYSFEAGKGASRLKACTCICRKEKKFYNVAEEIQSLKQRIMDISRKRETYGITNINYNSGERPSNQVTTLRRTTSYVDEQDYIFVGFQDVVQTLLAQLLKAEPRRSVLSIYGMGGLGKTTLARKLYTSPDILNSFPTRAWICVSQEYNTMDLLRTIIKSIQGCAKETLDLLEKMAEIDLENHLRDLLKECKYLVVVDDVWQREAWESLKRAFPDGKNGSRVIITTRKEDVAERVDHRGFVHKLRFLSQEESWDLFRRKLLDVRAMVPEMESLAKDMVEKCRGLPLAIVVLSGLLSHKKGLNQWQKVKDHLWKNIKEDKSIEISNILSLSYNDLSTALKQCFLYFGIFPEDQVVKADDIIRLWMAEGFIPRGEERMEDVADGFLNELIRRSLVQVAKTFWEKVTDCRVHDLLRDLAIQKALEVNFFDVYGPRSHSISSLCIRHGIHSEGERYLSSLDLSNLKLRSIMFFDPDFRKMSHINLRSEFQHLYVLYLDTNFGYVSMVPDAIGSLYHLKLLRLRGIHDIPSSIGNLKNLQTLVVVNGYTFFCQLPCKTADLINLRHLVVQYSEPLKCINKLTSLQVLDGVACDQWKDVDPVDLVNLRELSMDRIRSSYSLNNISSLKNLSTLKLICGERQSFASLEFVNCCEKLQKLWLQGRIEELPHLFSNSITMMVLSFSELTEDPMPILGRFPNLRNLKLDGAYEGKEIMCSDNSFSQLEFLHLRDLWKLERWDLGTSAMPLIKGLGIHNCPNLKEIPERMKDMELLKRNYML

SEQ ID NO: 58 - DNA, 852 bp, Solanum tuberosum
  exon (1)..(102)
  Intron (103)..(606)
  exon (607)..(738)
  3′UTR (742)..(852)
atgggttccaaggcaattatgtttcttggtctttttttggctattttcttaatgataagctctgaggttgctgctagggagttggcagctgagacttccaatggtaagcatttcgtttcatttgttccctctcttagggtgtgttcgataaaaaaaatgtttttcatgaaaaaaaatttgtaagaaaataagtgacttttttatttacatttttgtaaaataattggaaacgaaatcccaaaatttcaattaataatgatatagtttgatcataacacttaatatacattccttttgtttcattttatatgtctcatttactatgcataaagtttaagaatataccataaactttccaatctttgctaaactaaactaaaggtgtgtaaattatattataaatgtcctaagaattttgtgatcttatgttacattgaaattaaaaaatttacaattaaatatatattttaggaaaaggagtactcattttgaaatataataaaaaggaaagtacgacatataaattgaagctcaattcagagactagtcatgactcatatcgtacgtgttagctagtttaattaatcttcacaattcatcgatgcagcggtaaacgttgatggacattatcatggtggcggctatggtaagcactatggaaaacctaagaaatgctatagatgccacaaaaaatactgctgctcttatgaagaatatgtggctgaccagactcacaactaaataattaattgtgtgtattattattgtaaacttttgaaattaagtgacaagataataataatcttgctacttaagaccctttgcttgtaacaagtatgaataaagccattc

SEQ ID NO: 59 - protein, 78 aa, Solanum tuberosum
MGSKAIMFLGLFLAIFLMISSEVAARELAAETSNAVNVDGHYHGGGYGKHYGKPKKCYRCHKKYCCSYEEYVADQTHN

SEQ ID NO: 60 - DNA, 2913 bp, Solanum stoloniferum
  CDS (1)..(2913)
atggctgaagctttcattcaagttctgttagacaatctcacttctttcctcaaaggggaacttacattgcttttcggttttcaagatgagttccaaaggctttcaagcatgttttctacaatccaagccgtccttgaagatgctcaggagaagcaactcaacaacaagcctctagaaaattggttgcaaaaactcaatgctgctacatacgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggcaaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggtgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgataaggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatataattaa

SEQ ID NO: 61 - protein, 970 aa, Solanum stoloniferum
MAEAFIQVLLDNLTSFLKGELTLLFGFQDEFQRLSSMFSTIQAVLEDAQEKQLNNKPLENWLQKLNAATYEVDDILDEYKTKATRFSQSEYGRYHPKVIPFRHKVGKRMDQVMKKLKAIAEERKNFHLHEKIVERQAVRRETGSVLTEPQVYGRDKEKDEIVKILINNVSDAQHLSVLPILGMGGLGKTTLAQMVFNDQRVTEHFHSKIWICVSEDFDEKRLIKAIVESIEGRPLLGEMDLAPLQKKLQELLNGKRYLLVLDDVWNEDQQKWANLRAVLKVGASGASVLTTTRLEKVGSIMGTLQPYELSNLSQEDCWLLFMQRAFGHQEEINPNLVAIGKEIVKKSGGVPLAAKTLGGILCFKREERAWEHVRDSPIWNLPQDESSILPALRLSYHQLPLDLKQCFAYCAVFPKDAKMEKEKLISLWMAHGFLLSKGNMELEDVGDEVWKELYLRSFFQEIEVKDGKTYFKMHDLIHDLATSLFSANTSSSNIREINKHSYTHMMSIGFAEVVFFYTLPPLEKFISLRVLNLGDSTFNKLPSSIGDLVHLRYLNLYGSGMRSLPKQLCKLQNLQTLDLQYCTKLCCLPKETSKLGSLRNLLLDGSQSLTCMPPRIGSLTCLKTLGQFVVGRKKGYQLGELGNLNLYGSIKISHLERVKNDKDAKEANLSAKGNLHSLSMSWNNFGPHIYESEEVKVLEALKPHSNLTSLKIYGFRGIHLPEWMNHSVLKNIVSILISNFRNCSCLPPFGDLPCLESLELHWGSADVEYVEEVDIDVHSGFPTRIRFPSLRKLDIWDFGSLKGLLKKEGEEQFPVLEEMIIHECPFLTLSSNLRALTSLRICYNKVATSFPEEMFKNLANLKYLTISRCNNLKELPTSLASLNALKSLKIQLCCALESLPEEGLEGLSSLTELFVEHCNMLKCLPEGLQHLTTLTSLKIRGCPQLIKRCEKGIGEDWHKISHIPNVNIYN

SEQ ID NO: 62 - DNA, 2913 bp, Solanum patita
  CDS (1)..(2913)
atggctgaagctttcattcaagttctgttagacaatctcacttctttcctcaaaggggaacttacattgcttttcggttttcaagatgagttccaaaggctttcaagcatgttttctacaatccaagccgtccttgaagatgctcaggagaagcaactcaacaacaagcctctagaaaattggttgcaaaaactcaatgctgctacatacgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgatagggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagctgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatataattaa

SEQ ID NO: 63 - protein, 970 aa, Solanum patita
MAEAFIQVLLDNLTSFLKGELTLLFGFQDEFQRLSSMFSTIQAVLEDAQEKQLNNKPLENWLQKLNAATYEVDDILDEYKTKATRFSQSEYGRYHPKVIPFRHKVGKRMDQVMKKLKAIAEERKNFHLHEKIVERQAVRRETGSVLTEPQVYGRDKEKDEIVKILINNVSDAQHLSVLPILGMGGLGKTTLAQMVFNDQRVTEHFHSKIWICVSEDFDEKRLIKAIVESIEGRPLLGEMDLAPLQKKLQELLNGKRYLLVLDDVWNEDQQKWANLRAVLKVGASGASVLTTTRLEKVGSIMGTLQPYELSNLSQEDCWLLFMQRAFGHQEEINPNLVAIGKEIVKKSGGVPLAAKTLGGILCFKREERAWEHVRDSPIWNLPQDESSILPALRLSYHQLPLDLKQCFAYCAVFPKDAKMEKEKLISLWMAHGFLLSKGNMELEDVGDEVWKELYLRSFFQEIEVKDGKTYFKMHDLIHDLATSLFSANTSSSNIREINKHSYTHMMSIGFAEVVFFYTLPPLEKFISLRVLNLGDSTFNKLPSSIGDLVHLRYLNLYGSGMRSLPKQLCKLQNLQTLDLQYCTKLCCLPKETSKLGSLRNLLLDGSQSLTCMPPRIGSLTCLKTLGQFVVGRKKGYQLGELGNLNLYGSIKISHLERVKNDRDAKEANLSAKGNLHSLSMSWNNFGPHIYESEEVKVLEALKPHSNLTSLKIYGFRGIHLPEWMNHSVLKNIVSILISNFRNCSCLPPFGDLPCLESLELHWGSADVEYVEEVDIDVHSGFPTRIRFPSLRKLDIWDFGSLKGLLKKEGEEQFPVLEELIIHECPFLTLSSNLRALTSLRICYNKVATSFPEEMFKNLANLKYLTISRCNNLKELPTSLASLNALKSLKIQLCCALESLPEEGLEGLSSLTELFVEHCNMLKCLPEGLQHLTTLTSLKIRGCPQLIKRCEKGIGEDWHKISHIPNVNIYN

SEQ ID NO: 64 - DNA, 2913 bp, Solanum bulbocastanum
  CDS (1)..(2913)
atggctgaagctttcattcaagttctgctagacaatctcacttctttcctcaaaggggaacttgtattgcttttcggttttcaagatgagttccaaaggctttcaagcatgttttctacaattcaagccgtccttgaagatgctcaggagaagcaactcaacaacaagcctctagaaaattggttgcaaaaactcaatgctgctacatatgaagtcgatgacatcttggatgaatataaaaccaaggccacaagattctcccagtctgaatatggccgttatcatccaaaggttatccctttccgtcacaaggtcgggaaaaggatggaccaagtgatgaaaaaactaaaggcaattgctgaggaaagaaagaattttcatttgcacgaaaaaattgtagagagacaagctgttagacgggaaacaggttctgtattaaccgaaccgcaggtttatggaagagacaaagagaaagatgagatagtgaaaatcctaataaacaatgttagtgatgcccaacacctttcagtcctcccaatacttggtatggggggattaggaaaaacgactcttgcccaaatggtcttcaatgaccagagagttactgagcatttccattccaaaatatggatttgtgtctcggaagattttgatgagaagaggttaataaaggcaattgtagaatctattgaaggaaggccactacttggtgagatggacttggctccacttcaaaagaagcttcaggagttgctgaatggaaaaagatacttgcttgtcttagatgatgtttggaatgaagatcaacagaagtgggctaatttaagagcagtcttgaaggttggagcaagtggtgcttctgttctaaccactactcgtcttgaaaaggttggatcaattatgggaacattgcaaccatatgaactgtcaaatctgtctcaagaagattgttggttgttgttcatgcaacgtgcatttggacaccaagaagaaataaatccaaaccttgtggcaatcggaaaggagattgtgaaaaaaagtggtggtgtgcctctagcagccaaaactcttggaggtattttgtgcttcaagagagaagaaagagcatgggaacatgtgagagacagtccgatttggaatttgcctcaagatgaaagttctattctgcctgccctgaggcttagttaccatcaacttccacttgatttgaaacaatgctttgcgtattgtgcggtgttcccaaaggatgccaaaatggaaaaagaaaagctaatctctctctggatggcgcatggttttcttttatcaaaaggaaacatggagctagaggatgtgggcgatgaagtatggaaagaattatacttgaggtcttttttccaagagattgaagttaaagatggtaaaacttatttcaagatgcatgatctcatccatgatttggcaacatctctgttttcagcaaacacatcaagcagcaatatccgtgaaataaataaacacagttacacacatatgatgtccattggtttcgccgaagtggtgtttttttacactcttccccccttggaaaagtttatctcgttaagagtgcttaatctaggtgattcgacatttaataagttaccatcttccattggagatctagtacatttaagatacttgaacctgtatggcagtggcatgcgtagtcttccaaagcagttatgcaagcttcaaaatctgcaaactcttgatctacaatattgcaccaagctttgttgtttgccaaaagaaacaagtaaacttggtagtctccgaaatcttttacttgatggtagccagtcattgacttgtatgccaccaaggataggatcattgacatgccttaagactctaggtcaatttgttgttggaaggaagaaaggttatcaacttggtgaactaggaaacctaaatctctatggctcaattaaaatctcgcatcttgagagagtgaagaatgataaggacgcaaaagaagccaatttatctgcaaaagggaatctgcattctttaagcatgagttggaataactttggaccacatatatatgaatcagaagaagttaaagtgcttgaagccctcaaaccacactccaatctgacttctttaaaaatctatggcttcagaggaatccatctcccagagtggatgaatcactcagtattgaaaaatattgtctctattctaattagcaacttcagaaactgctcatgcttaccaccctttggtgatctgccttgtctagaaagtctagagttacactgggggtctgcggatgtggagtatgttgaagaagtggatattgatgttcattctggattccccacaagaataaggtttccatccttgaggaaacttgatatatgggactttggtagtctgaaaggattgctgaaaaaggaaggagaagagcaattccctgtgcttgaagagatgataattcacgagtgcccttttctgaccctttcttctaatcttagggctcttacttccctcagaatttgctataataaagtagctacttcattcccagaagagatgttcaaaaaccttgcaaatctcaaatacttgacaatctctcggtgcaataatctcaaagagctgcctaccagcttggctagtctgaatgctttgaaaagtctaaaaattcaattgtgttgcgcactagagagtctccctgaggaagggctggaaggtttatcttcactcacagagttatttgttgaacactgtaacatgctaaaatgtttaccagagggattgcagcacctaacaaccctcacaagtttaaaaattcggggatgtccacaactgatcaagcggtgtgagaagggaataggagaagactggcacaaaatttctcacattcctaatgtgaatatatatatttaa

SEQ ID NO: 65 - protein, 970 aa, Solanum bulbocastanum
MAEAFIQVLLDNLTSFLKGELVLLFGFQDEFQRLSSMFSTIQAVLEDAQEKQLNNKPLENWLQKLNAATYEVDDILDEYKTKATRFSQSEYGRYHPKVIPFRHKVGKRMDQVMKKLKAIAEERKNFHLHEKIVERQAVRRETGSVLTEPQVYGRDKEKDEIVKILINNVSDAQHLSVLPILGMGGLGKTTLAQMVFNDQRVTEHFHSKIWICVSEDFDEKRLIKAIVESIEGRPLLGEMDLAPLQKKLQELLNGKRYLLVLDDVWNEDQQKWANLRAVLKVGASGASVLTTTRLEKVGSIMGTLQPYELSNLSQEDCWLLFMQRAFGHQEEINPNLVAIGKEIVKKSGGVPLAAKTLGGILCFKREERAWEHVRDSPIWNLPQDESSILPALRLSYHQLPLDLKQCFAYCAVFPKDAKMEKEKLISLWMAHGFLLSKGNMELEDVGDEVWKELYLRSFFQEIEVKDGKTYFKMHDLIHDLATSLFSANTSSSNIREINKHSYTHMMSIGFAEVVFFYTLPPLEKFISLRVLNLGDSTFNKLPSSIGDLVHLRYLNLYGSGMRSLPKQLCKLQNLQTLDLQYCTKLCCLPKETSKLGSLRNLLLDGSQSLTCMPPRIGSLTCLKTLGQFVVGRKKGYQLGELGNLNLYGSIKISHLERVKNDKDAKEANLSAKGNLHSLSMSWNNFGPHIYESEEVKVLEALKPHSNLTSLKIYGFRGIHLPEWMNHSVLKNIVSILISNFRNCSCLPPFGDLPCLESLELHWGSADVEYVEEVDIDVHSGFPTRIRFPSLRKLDIWDFGSLKGLLKKEGEEQFPVLEEMIIHECPFLTLSSNLRALTSLRICYNKVATSFPEEMFKNLANLKYLTISRCNNLKELPTSLASLNALKSLKIQLCCALESLPEEGLEGLSSLTELFVEHCNMLKCLPEGLQHLTTLTSLKIRGCPQLIKRCEKGIGEDWHKISHIPNVNIYI

SEQ ID NO: 66 - DNA, 60 bp, Artificial Sequence
  Synthetic nucleotide sequence T-DNA RB
gtttacccgccaatatatcctgtcaggtaccggcgcgccaagcttgcatgcctgcaggtc

SEQ ID NO: 67 - DNA, 31 bp, Artificial Sequence
  Synthetic nucleotide sequence T-DNA RB P-273-1
ccggcgcgccaagcttgcatgcctgcaggtc

SEQ ID NO: 68 - DNA, 38 bp, Artificial Sequence
  Synthetic nucleotide sequence T-DNA RB P-288-5
tcaggtaccggcgcgccaagcttgcatgcctgcaggtc

SEQ ID NO: 69 - DNA, 30 bp, Artificial Sequence
  Synthetic nucleotide sequence T-DNA RB P-314-7
cggcgcgccaagcttgcatgcctgcaggtc

SEQ ID NO: 70 - DNA, 33 bp, Artificial Sequence
  Synthetic nucleotide sequence T-DNA RB P-423-1
taccggcgcgccaagcttgcatgcctgcaggtc

SEQ ID NO: 71 - DNA, 54 bp, Artificial Sequence
  Synthetic nucleotide sequence T-DNA LB
ttttactatttaattaaggatcctctagagtttacaccacaatatatcctgcca

SEQ ID NO: 72 - DNA, 49 bp, Artificial Sequence
  Synthetic nucleotide sequence T-DNA LB P-288-5
ttttactatttaattaaggatcctctagagtttacaccacaatatatcc

SEQ ID NO: 73 - DNA, 51 bp, Artificial Sequence
  Synthetic nucleotide sequence T-DNA LB P-315-3
ttttactatttaattaaggatcctctagagtttacaccacaatatatcctg

SEQ ID NO: 74 - DNA, 25 bp, Artificial Sequence
  Synthetic nucleotide sequence T-DNA LB P-415-4
ttttactatttaattaaggatcctc

SEQ ID NO: 75 - DNA, 28 bp, Artificial Sequence
  Synthetic nucleotide sequence T-DNA LB P-423-1
ttttactatttaattaaggatcctctag

SEQ ID NO: 76 - DNA, 34 bp, Artificial Sequence
  Synthetic nucleotide sequence T-DNA LB P-437-2
ttttactatttaattaaggatcctctagagttta

SEQ ID NO: 77 - DNA, 237 bp, Solanum tuberosum
atgggttccaaggcaattatgtttcttggtctttttttggctattttcttaatgataagctctgaggttgctgctagggagttggcagctgagacttccaatgcggtaaacgttgatggacattatcatggtggcggctatggtaagcactatggaaaacctaagaaatgctatagatgccacaaaaaatactgctgctcttatgaagaatatgtggctgaccagactcacaactaa